(19) United States
(12) Patent Application Publication
(10) Pub. No.: US 2025/0073023 A1
(43) Pub. Date: Mar. 6, 2025

(54) TAVI Deployment Accuracy - Stent Frame Improvements

(71) Applicant: St. Jude Medical, Cardiology Division, Inc., St. Paul, MN (US)

(72) Inventors: Peter J. Ness, Minneapolis, MN (US); Michael Morrissey, St. Paul, MN (US); Victoria Schuman, Minneapolis, MN (US); Jaron J. Olsoe, Minneapolis, MN (US); Heath Marnach, Minneapolis, MN (US)

(73) Assignee: St. Jude Medical, Cardiology Division, Inc., St. Paul, MN (US)

(21) Appl. No.: 18/810,994

(22) Filed: Aug. 21, 2024

Related U.S. Application Data
(60) Provisional application No. 63/579,378, filed on Aug. 29, 2023.

(51) Int. Cl. A61F 2/24

(52) U.S. Cl. CPC A61F 2/2418; A61F 2/2433; A61F 2220/0033; A61F 2230/0019; A61F 2230/0023; A61F 2250/0037

(57) ABSTRACT

A prosthetic heart valve may include a balloon-expandable frame extending between an inflow end and an outflow end, a plurality of prosthetic leaflets mounted within the frame, and an inner skirt positioned between the plurality of prosthetic leaflets and the frame. The frame may include a first row of diamond-shaped cells at the inflow end of the frame, a second row of diamond-shaped cells, and a row of outflow cells positioned at the outflow end of the frame. Each cell in the row of outflow cells may not be diamond-shaped, and, in an expanded condition of the frame, each cell in the row of outflow cells may have a larger area than that of each cell in the first and second rows of diamond-shaped cells. Each outflow cell may be at least partially defined by a commissure attachment feature, and each outflow cell may lack symmetry.

### CROSS-REFERENCE TO RELATED APPLICATIONS

This application claims the benefit of priority of U.S. Provisional Patent Application No. 63/579,378, filed Aug. 29, 2023, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves, including surgical heart valves and collapsible/expandable heart valves intended for transcatheter aortic valve replacement or implantation (“TAVR” or “TAVI”) or transcatheter mitral valve replacement (“TMVR”), are well known in the patent literature. Surgical or mechanical heart valves may be sutured into a native annulus of a patient during an open-heart surgical procedure, for example. Collapsible/expandable heart valves may be delivered into a patient via a tube-like delivery apparatus such as a catheter, a trocar, a laparoscopic instrument, or the like to avoid a more invasive procedure such as full open-chest, open-heart surgery. As used herein, reference to a “collapsible/expandable” heart valve includes heart valves that are formed with a small cross-section that enables them to be delivered into a patient through a tube-like delivery apparatus in a minimally invasive procedure, and then expanded to an operable state once in place, as well as heart valves that, after construction, are first collapsed to a small cross-section for delivery into a patient and then expanded to an operable size once in place in the valve annulus.

Collapsible/expandable prosthetic heart valves typically take the form of a one-way valve structure (often referred to as a valve assembly) mounted to/within an expandable stent (the terms “stent” and “frame” are used interchangeably herein). In general, these collapsible/expandable heart valves include a self-expanding or balloon-expandable stent, often made of nitinol or another shape-memory metal or metal alloy (for self-expanding stents) or steel or cobalt chromium (for balloon-expandable stents). Existing collapsible/expandable TAVR devices have been known to use different configurations of stent layouts—including straight vertical struts connected by “V”s as illustrated in U.S. Pat. No. 8,454,685, or diamond-shaped cell layouts as illustrated in U.S. Pat. No. 9,326,856, both of which are hereby incorporated herein by reference. The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

When expanding a prosthetic heart valve into the native heart valve annulus, accurate deployment is typically an important indicator of the success of the prosthesis. For example, for an aortic heart valve replacement, the position of the prosthesis relative to the aortic annulus, as well as the extent to which the prosthesis extends into the left ventricular outflow tract (“LVOT”), can impact performance attributes of the prosthesis such as hemodynamics, PV leak, and the necessity of implanting a pacemaker with the prosthetic heart valve. Thus, it would be desirable to be able to increase the accuracy with which the prosthetic heart valve can be placed within the native valve annulus to optimize performance attributes of the prosthetic heart valve.

### SUMMARY OF THE DISCLOSURE

According to one aspect of the disclosure, a prosthetic heart valve includes a balloon-expandable frame extending between an inflow end and an outflow end, a plurality of prosthetic leaflets mounted within the frame, and an inner skirt positioned between the plurality of prosthetic leaflets and the frame. The frame may include a first row of diamond-shaped cells at the inflow end of the frame, a second row of diamond-shaped cells, and a row of outflow cells positioned at the outflow end of the frame. Each cell in the row of outflow cells may not be diamond-shaped, and, in an expanded condition of the frame, each cell in the row of outflow cells may define an interior area that is larger than an interior area defined by each cell in the first and second rows of diamond-shaped cells. Each cell in the row of outflow cells may be at least partially defined by a commissure attachment feature, and each cell in the row of outflow cells may lack symmetry. The frame may include a third row of diamond-shaped cells positioned between the first and second rows of diamond-shaped cells. The first and second rows of diamond-shaped cells may include twelve cells each, and the row of outflow cells may include six cells. The frame may include an inflow section that includes the first and second rows of diamond-shaped cells, and an outflow section that includes the row of outflow cells, such that more force is required to expand the inflow section than the outflow section. The commissure attachment feature may include a plurality of commissure attachment features that each have a rectangular or triangular shape, and each of the commissure attachment features may be attached to one cell in the second row of diamond-shaped cells and may define portions of two cells in the outflow row of cells. The one cell in the second row of diamond-shaped cells may include two struts forming an outflow apex that is coupled to an inflow end of the corresponding commissure attachment feature. Each cell in the row of outflow cells may be defined by (i) a portion of one of the plurality of commissure attachment features, (ii) struts at an outflow end of a multiplicity of cells in the second row of diamond-shaped cells, and (iii) a plurality of linking struts extending between the outflow end of one of the multiplicity of cells in the second row of diamond-shaped cells and the one of the plurality of commissure attachment features. The plurality of linking struts may include a first linking strut extending in an outflow direction away from the one of the multiplicity of cells in the second row of diamond-shaped cells, a second linking strut extending from the first linking strut in a direction back toward the second row of diamond-shaped cells, and a third linking strut extending from the second linking strut towards, and connecting to, the one of the plurality of commissure attachment features. The third linking strut may be coupled to an outflow end of the one of the plurality of commissure attachment features. The first linking strut may have a width that is greater than a width of the second linking strut.

According to another aspect of the disclosure, a prosthetic heart valve may include a balloon-expandable frame extending between an inflow end and an outflow end, and a plurality of prosthetic leaflets mounted within the frame. The frame may include a first row of kite-shaped cells, and a second row of kite-shaped cells positioned in an outflow direction relative to the first row of kite-shaped cells. Each cell in the first row may be defined by two inflow struts and two outflow struts, each cell in the second row of kite-shaped cells is defined by two inflow struts and two outflow struts, the two outflow struts in the first row being the same as the two inflow struts in the second row. The two inflow struts of each cell in the first row may be wider than the two outflow struts of each cell in the first row or the two outflow struts of each cell in the second row. The two outflow struts of each cell in the first row may be wider than the two outflow struts of each cell in the second row. The two inflow struts of each cell in the first row may be thicker than the two outflow struts of each cell in the first row or the two outflow struts of each cell in the second row. The two outflow struts of each cell in the first row may be thicker than the two outflow struts of each cell in the second row. The two inflow struts in each cell in the first row may form a first angle relative to each other, and the two outflow struts in each cell in the first row may form a second angle relative to each other, the first angle being larger than the second angle. The two inflow struts in each cell in the second row may form a third angle relative to each other, and the two outflow struts in each cell in the second row may form a fourth angle relative to each other, the third angle being larger than the fourth angle, and the first angle being larger than the third angle.

According to another aspect of the disclosure, a method of implanting a prosthetic heart valve may include advancing a distal end of a delivery device to a native aortic valve while the prosthetic heart valve is crimped over a balloon on the distal end of the delivery device while the balloon is in an uninflated condition. Under fluoroscopy, it may be confirmed that an inflow end of a frame of the prosthetic heart valve is aligned with a desired target site while the balloon is in the uninflated condition. After confirming, inflation media may be passed into the balloon to inflate the balloon to expand the prosthetic heart valve into the native aortic valve. After expanding the prosthetic heart valve, the inflow end of the frame may be positioned at the desired target site. Between confirming and expanding the prosthetic heart valve, the delivery device may not be translated relative to the native aortic valve. During expanding the prosthetic heart valve, the frame of the prosthetic heart valve may axially foreshorten. During expanding the prosthetic heart valve, an outflow end of the frame may translate toward the target site while the inflow end of the frame does not translate relative to the target site. As the balloon inflates to expand the prosthetic heart valve, there may be greater friction between the balloon and an inflow end of the prosthetic heart valve than there is between the balloon and an outflow end of the prosthetic heart valve.

### BRIEF DESCRIPTION OF DRAWINGS

FIG. 1A is a perspective view of a stent of a prosthetic heart valve according to an embodiment of the disclosure.

FIG. 1B is a schematic front view of a section of the stent of FIG. 1A.

FIG. 1C is a schematic front view of a section of a stent according to an alternate embodiment of the prosthetic heart valve of FIG. 1A.

FIGS. 1D-E are front views of the stent section of FIG. 1C in a collapsed and expanded state, respectively.

FIGS. 1F-G are side views of a portion of the stent according to the embodiment of FIG. 1C in a collapsed and expanded state, respectively.

FIG. 1H is a flattened view of the stent according to the embodiment of FIG. 1C, as if cut and rolled flat.

FIGS. 1I-J are front and side views, respectively, of a prosthetic heart valve including the stent of FIG. 1C.

FIG. 1K illustrates the view of FIG. 1H with an additional outer cuff provided on the stent.

FIG. 2A illustrates a prosthetic heart valve crimped over a balloon of a delivery device.

FIG. 2B is a schematic view of the balloon of FIG. 2A after having been inflated.

FIG. 3A illustrates a stent in a collapsed condition and positioned near a target deployment site.

FIG. 3B illustrates the stent of FIG. 3A after expanding.

FIG. 4A is a schematic front view of a section of a stent of a prosthetic heart valve.

FIG. 4B illustrates the stent of FIG. 4A a stent in a collapsed condition and positioned near a target deployment site.

FIG. 4C illustrates the stent of FIG. 4B after expanding.

FIG. 5 is a highly schematic front view of a section of a stent of a prosthetic heart valve.

FIGS. 6-23 are schematic front views of sections of frames that are alternative versions of the frame portion shown in FIG. 4A.

FIGS. 24A-B are enlarged views of a commissure attachment feature according to another aspect of the disclosure.

FIG. 25 is an enlarged view of a commissure attachment feature according to a further aspect of the disclosure.

FIGS. 26-30 are schematic front views of sections of frames that are additional alternative versions of the frame portion shown in FIG. 4A.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing the valve disclosed herein, however, it should be noted that the use of the valve is not limited to the intended position and orientation but may be deployed in any type of lumen or passageway. For example, although the prosthetic heart valve is described herein as a prosthetic aortic valve, the same or similar structures and features can be employed in other heart valves, such as the pulmonary valve, the mitral valve, or the tricuspid valve. Further, the term “proximal,” when used in connection with a delivery device or system, refers to a direction relatively close to the user of that device or system when being used as intended, while the term “distal” refers to a direction relatively far from the user of the device. In other words, the leading end of a delivery device or system is positioned distal to the trailing end of the delivery device or system, when being used as intended. As used herein, the terms “substantially,” “generally,” “approximately,” and “about” are intended to mean that slight deviations from absolute are included within the scope of the term so modified. As used herein, the stent may assume an “expanded state” and a “collapsed state,” which refer to the relative radial size of the stent.

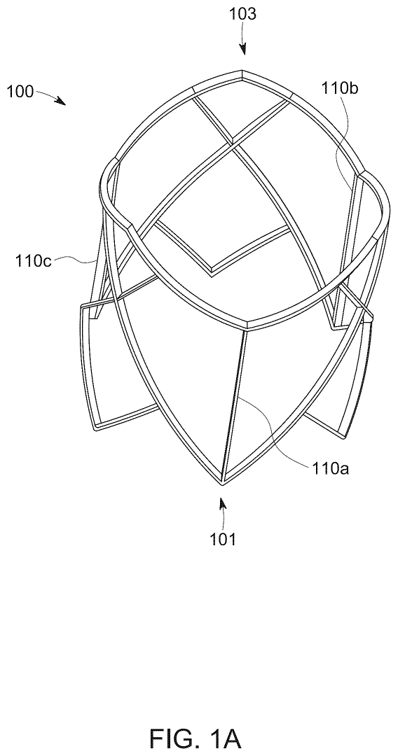

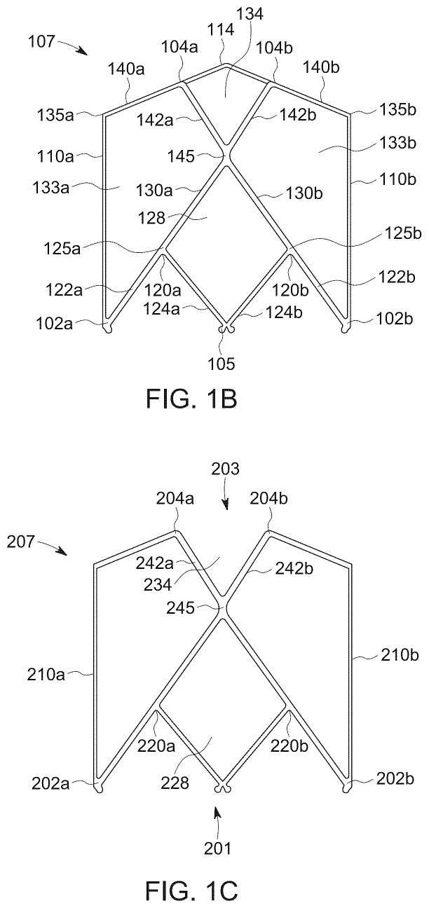

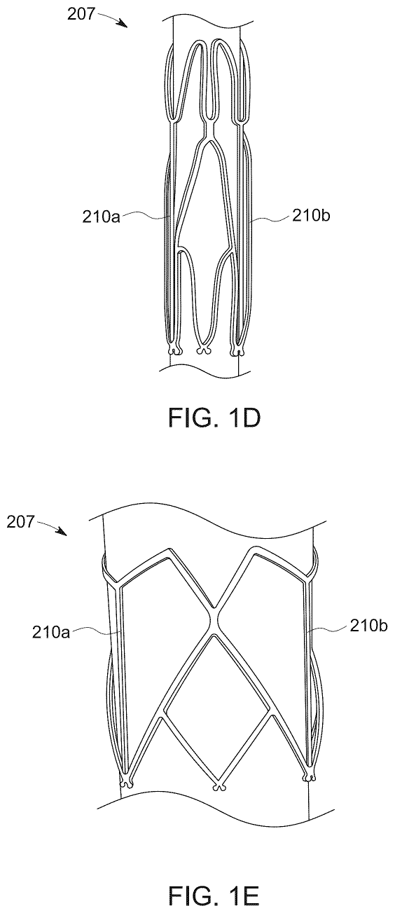

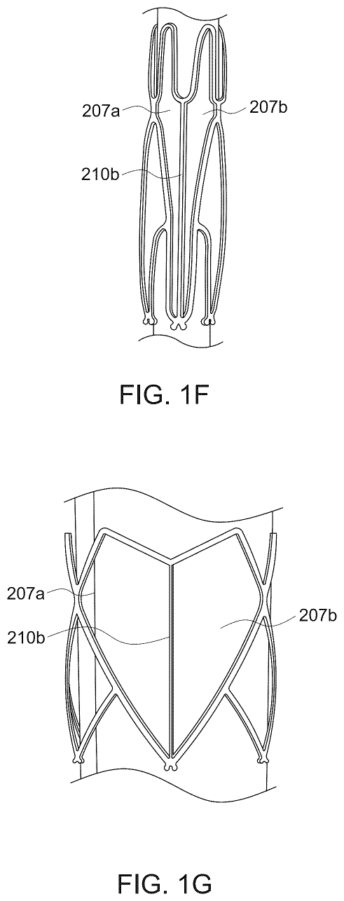

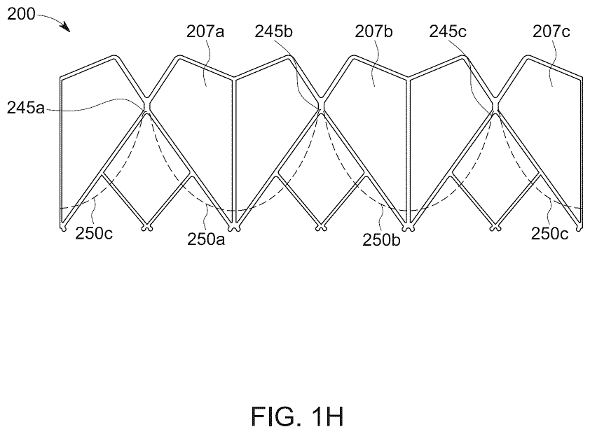

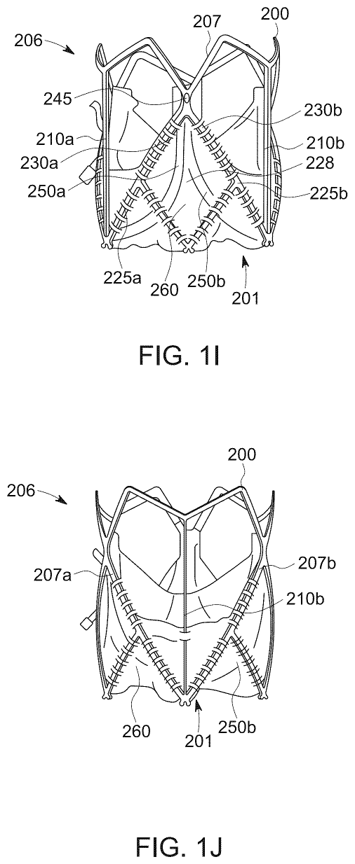

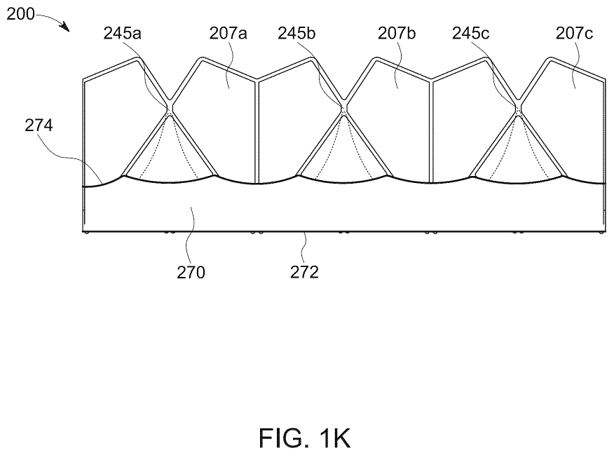

FIG. 1A illustrates a perspective view of a stent 100 of a prosthetic heart valve according to an embodiment of the disclosure. Stent 100 may include a frame extending in an axial direction between an inflow end 101 and an outflow end 103. Stent 100 includes three generally symmetric sections, wherein each section spans about 120 degrees around the circumference of stent 100. Stent 100 includes three vertical struts 110a, 110b, 110c, that extend in an axial direction substantially parallel to the direction of blood flow through the stent, which may also be referred to as a central longitudinal axis. Each vertical strut 110a, 110b, 110c may extend substantially the entire axial length between the inflow end 101 and the outflow end 103 of the stent 100 and may be disposed between and shared by two sections. In other words, each section is defined by the portion of stent 100 between two vertical struts. Thus, each vertical strut 110a, 110b, 110c is also separated by about 120 degrees around the circumference of stent 100. It should be understood that, if stent 100 is used in a prosthetic heart valve having three leaflets, the stent may include three sections as illustrated. However, in other embodiments, if the prosthetic heart valve has two leaflets, the stent may only include two of the sections.

FIG. 1B illustrates a schematic view of a stent section 107 of stent 100, which will be described herein in greater detail, and which is representative of all three sections. Stent section 107 depicted in FIG. 1B includes a first vertical strut 110a and a second vertical strut 110b. First vertical strut 110a extends axially between a first inflow node 102a and a first outer node 135a. Second vertical strut 110b extends axially between a second inflow node 102b and a second outer node 135b. As is illustrated, the vertical struts 110a, 110b may extend almost the entire axial length of stent 100. In some embodiments, stent 100 may be formed as an integral unit, for example by laser cutting the stent from a tube. The term “node” may refer to where two or more struts of the stent 100 meet one another. A pair of sequential inverted V's extends between inflow nodes 102a, 102b, which includes a first inflow inverted V 120a and a second inflow inverted V 120b coupled to each other at an inflow node 105. First inflow inverted V 120a comprises a first outer lower strut 122a extending between first inflow node 102a and a first central node 125a. First inflow inverted V 120a further comprises a first inner lower strut 124a extending between first central node 125a and inflow node 105. A second inflow inverted V 120b comprises a second inner lower strut 124b extending between inflow node 105 and a second central node 125b. Second inflow inverted V 120b further comprises a second outer lower strut 122b extending between second central node 125b and second inflow node 102b. Although described as inverted V's, these structures may also be described as half-cells, each half cell being a half-diamond cell with the open portion of the half-cell at the inflow end 101 of the stent 100.

Stent section 107 further includes a first central strut 130a extending between first central node 125a and an upper node 145. Stent section 107 also includes a second central strut 130b extending between second central node 125b and upper node 145. First central strut 130a, second central strut 130b, first inner lower strut 124a and second inner lower strut 124b form a diamond cell 128. Stent section 107 includes a first outer upper strut 140a extending between first outer node 135a and a first outflow node 104a. Stent section 107 further includes a second outer upper strut 140b extending between second outer node 135b and a second outflow node 104b. Stent section 107 includes a first inner upper strut 142a extending between first outflow node 104a and upper node 145. Stent section 107 further includes a second inner upper strut 142b extending between upper node 145 and second outflow node 104b. Stent section 107 includes an outflow inverted V 114 which extends between first and second outflow nodes 104a, 104b. First vertical strut 110a, first outer upper strut 140a, first inner upper strut 142a, first central strut 130a and first outer lower strut 122a form a first generally kite-shaped cell 133a. Second vertical strut 110b, second outer upper strut 140b, second inner upper strut 142b, second central strut 130b and second outer lower strut 122b form a second generally kite-shaped cell 133b. First and second kite-shaped cells 133a, 133b are symmetric and opposite each other on stent section 107. Although the term “kite-shaped,” is used above, it should be understood that such a shape is not limited to the exact geometric definition of kite-shaped. Outflow inverted V 114, first inner upper strut 142a and second inner upper strut 142b form upper cell 134. Upper cell 134 is generally kite-shaped and axially aligned with diamond cell 128 on stent section 107. It should be understood that, although designated as separate struts, the various struts described herein may be part of a single unitary structure as noted above. However, in other embodiments, stent 100 need not be formed as an integral structure and thus the struts may be different structures (or parts of different structures) that are coupled together.

FIG. 1C illustrates a schematic view of a stent section 207 according to an alternate embodiment of the disclosure. Unless otherwise stated, like reference numerals refer to like elements of above-described stent 100 but within the 200-series of numbers. Stent section 207 is substantially similar to stent section 107, including inflow nodes 202a, 202b, vertical struts 210a, 210b, first and second inflow inverted V's 220a, 220b and outflow nodes 204a, 204b. The structure of stent section 207 departs from that of stent section 107 in that it does not include an outflow inverted V. The purpose of an embodiment having such structure of stent section 207 shown in FIG. 1C is to reduce the required force to expand the outflow end 203 of the stent 200, compared to stent 100, to promote uniform expansion relative to the inflow end 201. Outflow nodes 204a, 204b are connected by a properly oriented V formed by first inner upper strut 242a, upper node 245 and second inner upper strut 242b. In other words, struts 242a, 242b may form a half diamond cell 234, with the open end of the half-cell oriented toward the outflow end 203. Half diamond cell 234 is axially aligned with diamond cell 228. Adding an outflow inverted V coupled between outflow nodes 204a, 204b contributes additional material that increases resistance to modifying the stent shape and requires additional force to expand the stent. The exclusion of material from outflow end 203 decreases resistance to expansion on outflow end 203, which may promote uniform expansion of inflow end 201 and outflow end 203. In other words, the inflow end 201 of stent 200 does not include continuous circumferential structure, but rather has mostly or entirely open half-cells with the open portion of the half-cells oriented toward the inflow end 201, whereas most of the outflow end 203 includes substantially continuous circumferential structure, via struts that correspond with struts 140a, 140b. All else being equal, a substantially continuous circumferential structure may require more force to expand compared to a similar but open structure. Thus, the inflow end 101 of stent 100 may require more force to radially expand compared to the outflow end 103. By omitting inverted V 114, resulting in stent 200, the force required to expand the outflow end 203 of stent 200 may be reduced to an amount closer to the inflow end 201.

FIG. 1D shows a front view of stent section 207 in a collapsed state and FIG. 1E shows a front view of stent section 207 in an expanded state. It should be understood that stent 200 in FIGS. 1D-E is illustrated with an opaque tube extending through the interior of the stent, purely for the purpose of helping illustrate the stent, and which may represent a balloon over which the stent section 207 is crimped. As described above, a stent comprises three symmetric sections, each section spanning about 120 degrees around the circumference of the stent. Stent section 207 illustrated in FIGS. 1D-E is defined by the region between vertical struts 210a, 210b. Stent section 207 is representative of all three sections of the stent. Stent section 207 has an arcuate structure such that when three sections are connected, they form one complete cylindrical shape. FIGS. 1F-G illustrate a portion of the stent from a side view. In other words, the view of stent 200 in FIGS. 1F-G is rotated about 60 degrees compared to the view of FIGS. 1D-E. The view of the stent depicted in FIGS. 1F-G is centered on vertical strut 210b showing approximately half of each of two adjacent stent sections 207a, 207b on each side of vertical strut 210b. Sections 207a, 207b surrounding vertical strut 210b are mirror images of each other. FIG. 1F shows stent sections 207a, 207b in a collapsed state whereas FIG. 1G shows stent sections 207a, 207b in an expanded state.

FIG. 1H illustrates a flattened view of stent 200 including three stent sections 207a, 207b, 207c, as if the stent has been cut longitudinally and laid flat on a table. As depicted, sections 207a, 207b, 207c are symmetric to each other and adjacent sections share a common vertical strut. As described above, stent 200 is shown in a flattened view, but each section 207a, 207b, 207c has an arcuate shape spanning 120 degrees to form a full cylinder. Further depicted in FIG. 1H are leaflets 250a, 250b, 250c coupled to stent 200. However, it should be understood that only the connection of leaflets 250a-c is illustrated in FIG. 1H. In other words, each leaflet 250a-c would typically include a free edge, with the free edges acting to coapt with one another to prevent retrograde flow of blood through the stent 200, and the free edges moving radially outward toward the interior surface of the stent to allow antegrade flow of blood through the stent. Those free edges are not illustrated in FIG. 1H. Rather, the attached edges of the leaflets 250a-c are illustrated in dashed lines in FIG. 1H. Although the attachment may be via any suitable modality, the attached edges may be preferably sutured to the stent 200 and/or to an intervening cuff or skirt between the stent and the leaflets 250a-c. Each of the three leaflets 250a, 250b, 250c, extends about 120 degrees around stent 200 from end to end and each leaflet includes a belly that may extend toward the radial center of stent 200 when the leaflets are coapted together. Each leaflet extends between the upper nodes of adjacent sections. First leaflet 250a extends from first upper node 245a of first stent section 207a to second upper node 245b of second stent section 207b. Second leaflet 250b extends from second upper node 245b to third upper node 245c of third stent section 207c. Third leaflet 250c extends from third upper node 245c to first upper node 245a. As such, each upper node includes a first end of a first leaflet and a second end of a second leaflet coupled thereto. In the illustrated embodiment, each end of each leaflet is coupled to its respective node by suture. However, any coupling means may be used to attach the leaflets to the stent. It is further contemplated that the stent may include any number of sections and/or leaflets. For example, the stent may include two sections, wherein each section extends 180 degrees around the circumference of the stent. Further, the stent may include two leaflets to mimic a bicuspid valve. Further, it should be noted that each leaflet may include tabs or other structures (not illustrated) at the junction between the free edges and attached edges of the leaflets, and each tab of each leaflet may be coupled to a tab of an adjacent leaflet to form commissures. In the illustrated embodiment, the leaflet commissures are illustrated attached to nodes where struts intersect. However, in other embodiments, the stent 200 may include commissure attachment features built into the stent to facilitate such attachment. For example, commissures attachment features may be formed into the stent 200 at nodes 245a-c, with the commissure attachment features including one or more apertures to facilitate suturing the leaflet commissures to the stent. Further, leaflets 250a-c may be formed of a biological material, such as animal pericardium, or may otherwise be formed of synthetic materials, such as plastics, fabrics, and/or polymers, including ultra-high molecular weight polyethylene (UHMWPE).

FIGS. 1I-J illustrate prosthetic heart valve 206, which includes stent 200, a cuff 260 coupled to stent 200 (for example via sutures) and leaflets 250a, 250b, 250c attached to stent 200 and/or cuff 260 (for example via sutures). Prosthetic heart valve 206 is intended for use in replacing an aortic valve, although the same or similar structures may be used in a prosthetic valve for replacing other heart valves. Cuff 260 is disposed on a luminal or interior surface of stent 200, although the cuff could be disposed alternately or additionally on an abluminal or exterior surface of the stent. The cuff 260 may include an inflow end disposed substantially along inflow end 201 of stent 200. FIG. 1I shows a front view of valve 206 showing one stent portion 207 between vertical struts 210a, 210b including cuff 260 and an outline of two leaflets 250a, 250b sutured to cuff 260. Different methods of suturing leaflets to the cuff as well as the leaflets and/or cuff to the stent may be used, many of which are described in U.S. Pat. No. 9,326,856 which is hereby incorporated by reference. In the illustrated embodiment, the upper (or outflow) edge of cuff 260 is sutured to first central node 225a, upper node 245 and second central node 225b, extending along first central strut 230a and second central strut 230b. The upper (or outflow) edge of cuff 260 continues extending approximately between the second central node of one section and the first central node of an adjacent section. Cuff 260 extends between upper node 245 and inflow end 201. Thus, cuff 260 covers the cells of stent portion 207 formed by the struts between upper node 245 and inflow end 201, including diamond cell 228. FIG. 1J illustrates a side view of stent 200 including cuff 260 and an outline of leaflet 250b. In other words, the view of valve 206 in FIG. 1J is rotated about 60 degrees compared to the view of FIG. 1I. The view depicted in FIG. 1J is centered on vertical strut 210b showing approximately half of each of two adjacent stent sections 207a, 207b on each side of vertical strut 210b. Sections 207a, 207b surrounding vertical strut 210b are mirror images of each other. As described above, the cuff may be disposed on the stent's interior or luminal surface, its exterior or abluminal surface, and/or on both surfaces. A cuff ensures that blood does not just flow around the valve leaflets if the valve or valve assembly are not optimally seated in a valve annulus. A cuff, or a portion of a cuff disposed on the exterior of the stent, can help minimize or eliminate leakage around the outside of the valve (the latter known as paravalvular leakage or “PV” leakage). In the embodiment illustrated in FIGS. 1I-J, the cuff 260 only covers about half of the axial extent of stent 200, leaving about half of the stent uncovered by the cuff. With this configuration, less cuff material is required compared to a cuff that covers more or all of the stent 200. Less cuff material may allow for the prosthetic heart valve 206 to crimp down to a smaller profile when collapsed. It is contemplated that the cuff may cover any amount of surface area of the cylinder formed by the stent. For example, the upper edge of the cuff may extend straight around the circumference of any cross section of the cylinder formed by the stent. Cuff 260 may be formed of any suitable material, including a biological material such as animal pericardium, or a synthetic material such as UHMWPE.

As noted above, FIGS. 1I-J illustrate a cuff 260 positioned on an interior of the stent 200. An example of an additional outer cuff 270 is illustrated in FIG. 1K. It should be understood that outer cuff 270 may take other shapes than that shown in FIG. 1K. The outer cuff 270 shown in FIG. 1K may be included without an inner cuff 260, but preferably is provided in addition to an inner cuff 260. The outer cuff 270 may be formed integrally with the inner cuff 260 and folded over (e.g. wrapped around) the inflow edge of the stent, or may be provided as a member that is separate from inner cuff 260. Outer cuff 270 may be formed of any of the materials described herein in connection with inner cuff 260. In the illustrated embodiment, outer cuff 270 includes an inflow edge 272 and an outflow edge 274. If the inner cuff 260 and outer cuff 270 are formed separately, the inflow edge 272 may be coupled to an inflow end of the stent 200 and/or an inflow edge of the inner cuff 260, for example via suturing, ultrasonic welding, or any other suitable attachment modality. The coupling between the inflow edge 272 of the outer cuff 270 and the stent 200 and/or inner cuff 260 preferably results in a seal between the inner cuff 260 and outer cuff 270 at the inflow end of the prosthetic heart valve so that any retrograde blood that flows into the space between the inner cuff 260 and outer cuff 270 is unable to pass beyond the inflow edges of the inner cuff 260 and outer cuff 270. The outflow edge 274 may be coupled at selected locations around the circumference of the stent 200 to struts of the stent 200 and/or to the inner cuff 260, for example via sutures. With this configuration, an opening may be formed between the inner cuff 260 and outer cuff 270 circumferentially between adjacent connection points, so that retrograde blood flow will tend to flow into the space between the inner cuff 260 and outer cuff 270 via the openings, without being able to continue passing beyond the inflow edges of the cuffs. As blood flows into the space between the inner cuff 260 and outer cuff 270, the outer cuff 270 may billow outwardly, creating even better sealing between the outer cuff 270 and the native valve annulus against which the outer cuff 270 presses. The outer cuff 270 may be provided as a continuous cylindrical member, or a strip that is wrapped around the outer circumference of the stent 200, with side edges, which may be parallel or non-parallel to a center longitudinal axis of the prosthetic heart valve, attached to each other so that the outer cuff 270 wraps around the entire circumference of the stent 200.

The stent may be formed from biocompatible materials, including metals and metal alloys such as cobalt chrome (or cobalt chromium) or stainless steel, although in some embodiments the stent may be formed of a shape memory material such as nitinol or the like. In some embodiments, the stent may be formed with cobalt chromium with additional metal or metal alloys such as nickel and/or molybdenum. The stent is thus configured to collapse upon being crimped to a smaller diameter and/or expand upon being forced open, for example via a balloon within the stent expanding, and the stent will substantially maintain the shape to which it is modified when at rest. The stent may be crimped to collapse in a radial direction and lengthen (to some degree) in the axial direction, reducing its profile at any given cross-section. The stent may also be expanded in the radial direction and foreshortened (to some degree) in the axial direction.

The prosthetic heart valve may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing the valve is inserted through the femoral artery and threaded against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

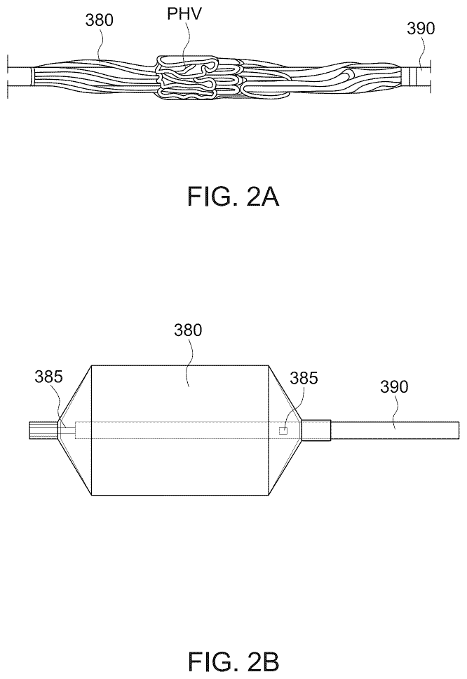

Referring to FIG. 2A, an example of a prosthetic heart valve PHV, which may include a stent similar to stents 100 or 200, is shown crimped over a balloon 380 of a balloon catheter 390 while the balloon 380 is in a deflated condition. It should be understood that other components of the delivery device, such as a handle used for steering and/or deployment, as well as a syringe for inflating the balloon 380, are omitted from FIGS. 2A-B. The prosthetic heart valve PHV may be delivered intravascularly, for example through the femoral artery, around the aortic arch, and into the native aortic valve annulus, while in the crimped condition shown in FIG. 2A. Once the desired position is obtained, fluid may be pushed through the balloon catheter 390 to inflate the balloon 380, as shown in FIG. 2B. FIG. 2B omits the prosthetic heart valve PHV, but it should be understood that, as the balloon 380 inflates, it forces the prosthetic heart valve PHV to expand into the native aortic valve annulus (although it should be understood that other heart valves may be replaced using the concepts described herein). In the illustrated example, fluid flows from a syringe (not shown) into the balloon 380 through a lumen within balloon catheter 390 and into one or more ports 385 located internal to the balloon 380. In the particular illustrated example of FIG. 2B, a first port 385 may be one or more apertures in a side wall of the balloon catheter 390, and a second port 385 may be the distal open end of the balloon catheter 390, which may terminate within the interior space of the balloon 380.

One potential complication with most expandable prosthetic heart valves is that typically, as the prosthetic heart valve radially expands into the native valve annulus during deployment, it also shortens axially. This is typically true of both self-expanding and balloon-expandable valves. The reason that this axial foreshortening may be problematic is that the axial position of certain valve elements relative to the native valve annulus prior to expansion will often not be the same as the axial position of those valve elements relative to the native valve annulus after expansion. In other words, if the prosthetic heart valve has a particular alignment that is shown under visualization (e.g. fluoroscopy) just prior to deployment, there will typically be at least some axial shifting of that relative alignment during deployment, and if such shifting is not minimized or otherwise compensated for, the resulting position of the deployed prosthetic heart valve may be non-optimal.

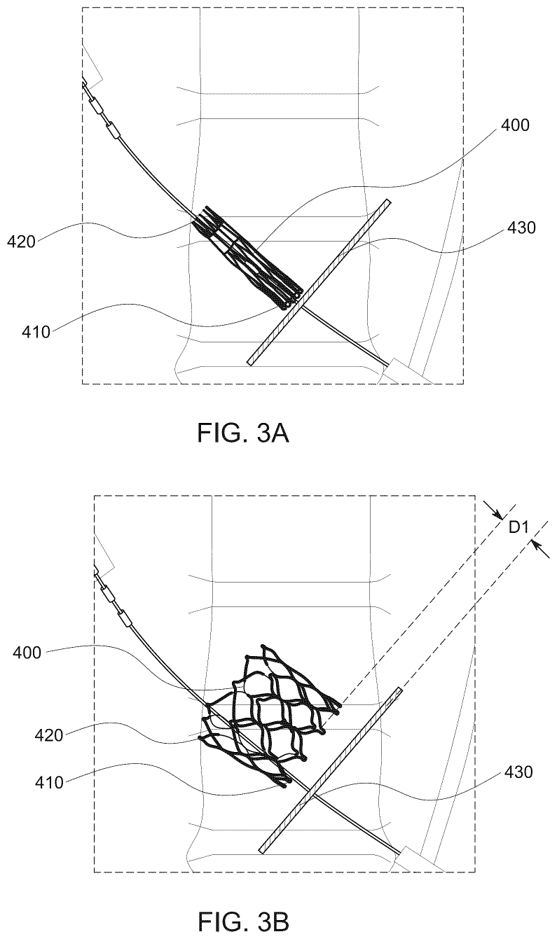

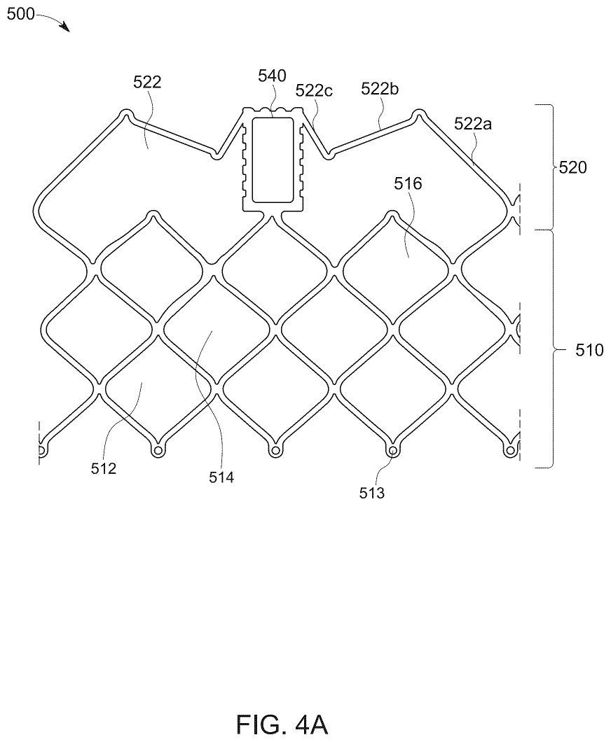

An example of the above-described axial shift is illustrated in FIGS. 3A-B. FIG. 3A illustrates a stent of a prosthetic heart valve collapsed over a balloon within a patient, with the figure being a fluoroscopic (e.g. x-ray) image so that only the metal stent 400 of the valve is easily visible. When deploying prosthetic heart valves, it is typically important that the inflow end of the prosthetic heart valve has a desired alignment with respect to the native valve annulus into which it is deployed. In FIGS. 3A-B, the desired target position of the inflow end 410 of the prosthetic heart valve is indicated by target line 430. The positioning of the outflow end 420 relative to the native valve annulus is typically less important (although not unimportant) compared to the alignment of the inflow end 410. In FIG. 3A, the stent 400 is still collapsed over a balloon and the inflow end 410 is exactly aligned with the target line 430. However, as deployment occurs, the stent 400 radially expands and axially foreshortens, so that after deployment, the inflow end 410 of the stent 400 is now a spaced distance D1 away from the target line 430. In other words, because axially foreshortening occurred, and because that axial foreshortening was not (1) minimized and/or (2) compensated for otherwise, the initial relative positioning of the stent 400 with the anatomy (while collapsed) was not a reliable indicator of the final positioning of the stent 400 relative to the anatomy (when expanded). One way to compensate for this change in positioning is to try to advance the inflow end 410 of the stent 400 beyond the target line 430 prior to deployment, with the hope that compensated distance will equal the axial shift of the inflow end 410 so that the stent 400 ends up in the desired position after deployment. However, this may not be a particularly reliable method and may lead to inconsistent results. It would be preferable to have features in the prosthetic heart valve itself and/or on the delivery device that result in more accurate placement of the prosthetic heart valve during deployment and minimize the need for subjective positioning compensation at the beginning of deployment.

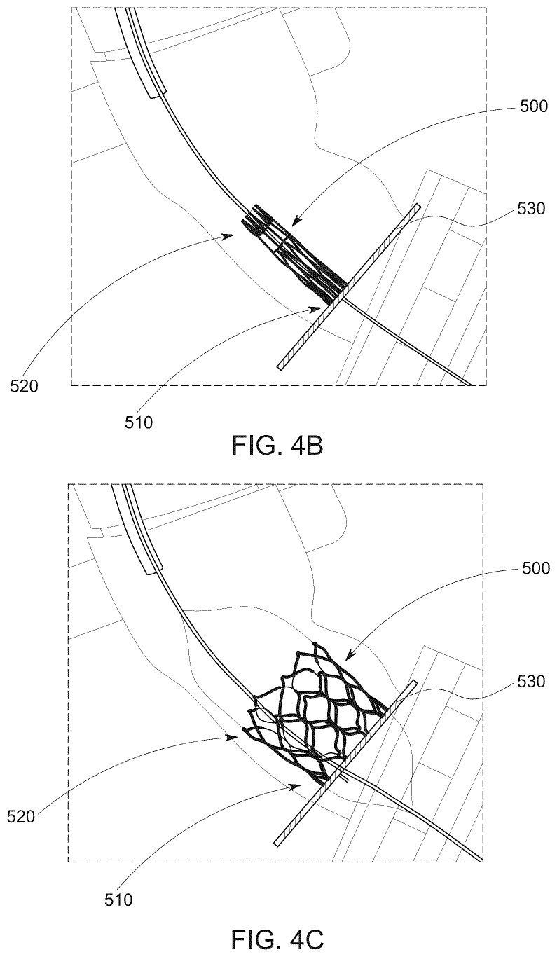

FIG. 4A illustrates a stent 500 for use as part of a balloon-expandable prosthetic heart valve that may be generally similar to prosthetic heart valve 206, but for the specific configuration of the stent 500. As with FIG. 1B, it should be understood that FIG. 4A illustrates only a section of a stent 500, and as if laid out flat on a table. In other words, the section of stent 500 in FIG. 4A may represent approximately one-third of a complete stent 500, particularly if stent 500 is used in conjunction with a three-leaflet prosthetic heart valve. In the illustrated embodiment, stent 500 is a balloon-expandable stent and may be formed of cobalt-chromium, which may (but need not) include additional materials such as nickel and/or molybdenum. As is described in greater detail below, stent 500 may include features that control the foreshortening and/or stability of the stent 500 during expansion so that at least some stent elements of the stent 500 are more predictable indicators of final implant position after foreshortening.

Generally, stent 500 includes stent geometries that tend to stabilize the inflow end of the stent 500 and/or increase retention of the inflow end of the stent 500 on the balloon 380, and which more readily allow for stent foreshortening (during radial expansion) at the outflow end of the stent 500. Referring to FIG. 4A, stent 500 may include an inflow section 510 and an outflow section 520. In the illustrated embodiment, the inflow section 510 may include a plurality of rows of generally diamond-shaped cells. For example, the inflow section 510 may include an inflow-most row of diamond-shaped cells 512, an adjacent intermediate row of diamond-shaped cells 514, and an outflow-most row of diamond-shaped cells 516. With this configuration, each cell in the intermediate row of cells 514 includes two struts that are shared with an adjacent cell in the inflow-most row of cells 512, and two struts that are shared with an adjacent cell in the outflow-most row of cells 516. It should be understood that although the term “outflow-most” is used in connection with the third row of cells 516, additional stent structure, described in more detail below, is still provided in the outflow direction relative to the outflow-most row of cells 516. An inflow apex of each cell in the first row of cells 512 may include an aperture 513 formed therein, which may accept sutures or similar features which may help couple other elements, such as an inner cuff, outer cuff, and/or prosthetic leaflets, to the stent 500. However, as is true of all other frame embodiments described herein, apertures similar to apertures 513 may optionally be included, or omitted, from the inflow apices of the inflow-most row of cells. In the illustrated embodiment, assuming that the stent 500 is for use with a three-leaflet valve and thus the section shown in FIG. 4A represents one-third of the stent, each row of cells 512, 514, 516 includes twelve individual cells. However, as is described in greater detail below, this cell number may be modified. In one particular example, the inflow section 510 may include only two rows of cells, with all of the cells being diamond-shaped. In another particular example, the inflow section 510 may include one or two rows of diamond-shaped cells, and one row of hexagonal-shaped cells, with the row of hexagonal-shaped cells being positioned at the inflow-most row of inflow section 510, at the outflow-most row of inflow section 510, or in a middle row of the inflow section 510 (if three rows are provided in the inflow section 510).

Still referring to FIG. 4A, the outflow section 520 of the stent 500 may include larger cells 522 that have shapes other than a regular polygon. For example, the lower part of the larger cells 522 may be defined by the two upper struts of a cell 516, and one upper strut of each of the two adjacent cells 516. In other words, the lower end of each larger cell 522 may be formed by a group of four consecutive upper struts of three circumferentially adjacent cells 516. The top of the larger cells 522 may be defined by three linking struts 522a, 522b, 522c. The first linking strut 522a may couple to a top or outflow apex of a cell 516 and extend upwards at an angle toward a commissure attachment feature (“CAF”) 540. The second linking strut 522b may extend from an end of the first linking strut 522a back downwardly at an angle toward the outflow-most row of cells 516. The third linking strut 522c may extend from an end of the second linking strut 522b back upwardly and connect directly to the CAF 540. To the extent that the larger cells 522 include sides, a first side is defined by a portion of the CAF 540, and a second side is defined by the connection between first linking strut 522a and the corresponding upper strut of the cell 516 attached to the first linking strut 522a.

The CAF 540 may generally serve as an attachment site for leaflet commissures (e.g. where two prosthetic leaflets join each other) to be coupled to the stent 500. In the illustrated example, the CAF 540 is generally rectangular and has a longer axial length than circumferential length. The CAF 540 also defines an interior open rectangular space. The struts that form CAF 540 may be generally smooth on the surface defining the open rectangular space, but some or all of the struts may have one or more suture notches on the opposite surfaces. For example, in the illustrated example, CAF 540 includes two side struts (on the longer side of the rectangle) and one top (or outflow) strut that all include alternating projections and notches on their exterior facing surfaces. These projections and notches may help maintain the position of one or more sutures that wrap around these struts. These sutures may directly couple the prosthetic leaflets to the frame 500, and/or may directly couple an intermediate sheet of material (e.g. fabric or tissue) to the CAF 540, with the prosthetic leaflets being directly coupled to that intermediate sheet of material. In some embodiments, tabs or ends of the prosthetic leaflets may be pulled through the opening of the CAF 540, but in other embodiments the prosthetic leaflets may remain mostly or entirely within the inner diameter of the frame 500. It should be understood that balloon-expandable frames, including all other embodiments described herein, are typically formed of metal or metal alloys that is very stiff, particularly in comparison to self-expanding valves. At least in part because of this stiffness, although the prosthetic leaflets may be sutured or otherwise directly coupled to the frame at the CAFs 540, it is preferably that most or all of the remaining portions of the prosthetic leaflets are not attached directly to the frame 500, but are rather attached directly to an inner skirt, which in turn is directly connected to the frame 500. Further, it should be understood that other shapes and configurations of CAFs 540 may be appropriate. Various other embodiments of frames are provided below, and it should be understood that features of one frame embodiment may be combined with features of other frame embodiments, as described in greater detail below.

With the embodiment described above, stent 500 includes three rows of diamond-shaped cells 512, 514, 516 and a single row of larger cells 522. In a three-leaflet embodiment of a prosthetic heart valve that incorporates stent 500, each row of diamond-shaped cells 512, 514, 516 includes twelve cells, while the row of larger cells includes six larger cells 522. As should be understood, the area defined by each individual cell 512, 514, 516 is significantly smaller than the area defined by each larger cell 522 when the stent 500 is expanded. There is also significantly more structure (e.g. struts) that create each row of individual cells 512, 514, 516 than structure that creates the row of larger cells 522.

One consequence of the above-described configuration is that the inflow section 510 has a higher cell density than the outflow section 520. In other words, the total numbers of cells, as well as the number of cells per row of cells, is larger in the inflow section 510 compared to the outflow section 520. The configuration of stent 500 described above may also result in the inflow section 510 being generally stiffer than the outflow section 520 and/or more radial force being required to expand the inflow section 510 compared to the outflow section 520, despite the fact that the stent 500 may be formed of the same metal or metal alloy throughout.

In addition to cell density, the width and/or thickness of the struts that form the cells of the inflow section 510 and outflow section 520 may be modified to adjust the radial force required to expand the stent frame 500. As used herein, strut “width” refers to the circumferential direction of the stent 500, while strut “thickness” refers to the depth dimension from the radially outer to radially inner surface (which may also be referred to was the “wall thickness” of the stent). For example, the strut “width” may generally refer to the left-to-right dimension visible in FIG. 4A for the struts, while the strut “thickness” may generally refer to the dimension into the page in the view of FIG. 4A. Increasing the strut thickness will typically increase the radial force required to expand the area of increased thickness (all else being equal) while increasing the strut width will also typically increase the radial force required to expand the area of increased width (all else being equal). For example, in the embodiment of the frame 500 of FIG. 4A, all of the cells in the rows 512, 514, 516 of the inflow section 510 have about the same strut width, which is larger than most or all of the struts forming the lager cells 522 in the outflow section 520. For example, as shown in FIG. 4A, linking strut 522 may have a width that is about equal or slightly smaller than the width of the struts forming cells in rows 512, 514, 516, while linking struts 522b and 522c may have a width that is even smaller than that of linking strut 522a. In some embodiments, each linking strut 522a, 522b, 522c may have a constant width, but in other embodiments, the linking struts may have tapering widths, for example with relatively large widths near the connection of linking strut 522a to a cell in outflow row 516, with the width of the linking strut 522a narrowing in a direction away from that connection. It should be understood that the strut widths and thicknesses may be modified in other ways than those explicitly shown and described to further adjust the amount of radial force required to expand the inflow section 510 versus the outflow section 520.

FIGS. 4B-C illustrate one of the benefits of varying the amount of radial force required to expand stent 500 at different locations along the axial extent thereof. 4B illustrates stent 500 of a prosthetic heart valve collapsed over a balloon within a patient, with the figure being a fluoroscopic (e.g. x-ray) image so that only the metal stent 500 of the valve is easily visible. Similar to FIGS. 3A-B, in FIGS. 4B-C the desired target position of the inflow edge of the inflow section 510 of the prosthetic heart valve is indicated by target line 530. In FIG. 4B, the stent 500 is still collapsed over a balloon and the inflow edge of the inflow section 510 is exactly aligned with the target line 530. As deployment occurs (and the balloon inflates), the stent 500 radially expands and axially foreshortens. However, unlike the situation of FIGS. 3A-B, the stent 500 of FIGS. 4B-C expands so that, after deployment, the inflow edge of the inflow section 510 of the stent 500 is still exactly (or nearly exactly) aligned with target line 530. This result is achievable because, as the balloon expands, the outflow section 520 of the stent will foreshorten significantly more easily than the inflow section 510 due, at least in part, to the differential radial forces required to expand the different stent sections. In other words, the inflow edge of the inflow section 510 of the stent 500 may be used, prior to expansion or deployment, as a reliable visual indicator of where the inflow edge of the inflow section 510 of the stent 500 will be positioned after expansion or deployment. This reduces or eliminates the need to otherwise compensate for the axial foreshortening, for example by advancing the stent 500 “beyond” the target line 530 pre-deployment to try to achieve correct final positioning of the inflow end of the stent 500 at the target line 530 after deployment. This may eliminate significant guesswork from the deployment procedure, and generally increase the accuracy of the final axial positioning of the stent relative to the native valve annulus after deployment.

It should be understood that, while FIG. 4A only shows one example of one section of a frame 500, the other components of the prosthetic heart valve that incorporates frame 500 may be similar or identical to those described in connection with prosthetic heart valve 206. For example, prosthetic leaflets, an interior cuff and/or exterior cuff similar to those shown and described in connection with prosthetic heart valve 206 may be used with frame 500 to create a prosthetic heart valve. And although one particular embodiment of frame 500 is shown, it should be understood that certain modifications could be made, such as increasing or decreasing the total number of rows of cells (for example in the inflow section 510) and/or increasing or decreasing the total number of cells per row (for example in the inflow section 510). Generally increasing the number of rows or the number of cells per row may increase the stiffness of that area of the frame (or require more force to expand that section), if all else remains equal, and vice versa. It should also be understood that the illustrated embodiment of FIG. 4A may provide additional benefits that are not fully related to variable forces. For example, the relatively large cells 522 in the outflow section 522 may be uncovered by an inner or outer cuff so that the interior of these large cells 522 remain open. After deployment, the large open space of cells 522 may help ensure satisfactory blood flow to the coronary arteries, and may also make later intervention into the coronary arteries easier. For example, if a stent needs to be placed in a coronary artery after a prosthetic heart valve that incorporates frame 500 is implanted, a catheter containing such a coronary stent may relatively easily pass through the interior space of cells 522 to reach the coronary arteries. This may be even more true if the prosthetic heart valve that incorporates stent 500 is implanted such that each of the three CAFs 540 are aligned with a native commissure of the native heart valve. In some embodiments, upon implantation, the coronary ostia may be generally aligned beyond the outflow end of the frame 500, for example near a valley between two adjacent linking struts 522a.

As with other stents described herein, stent 500 may be formed from laser cutting a continuous tube of the desired metal or metal alloy, so that the stent 500 is formed as a single integral member, although other methods may be used to form the stent 500, including ones in which the stent 500 is formed as multiple pieces that are joined together.

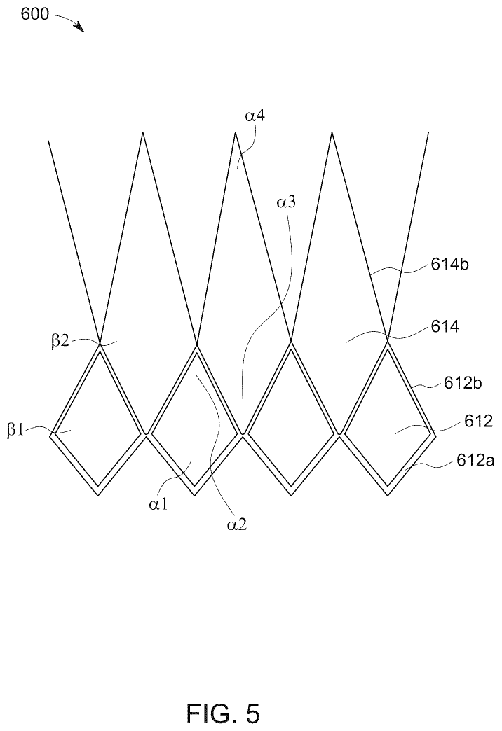

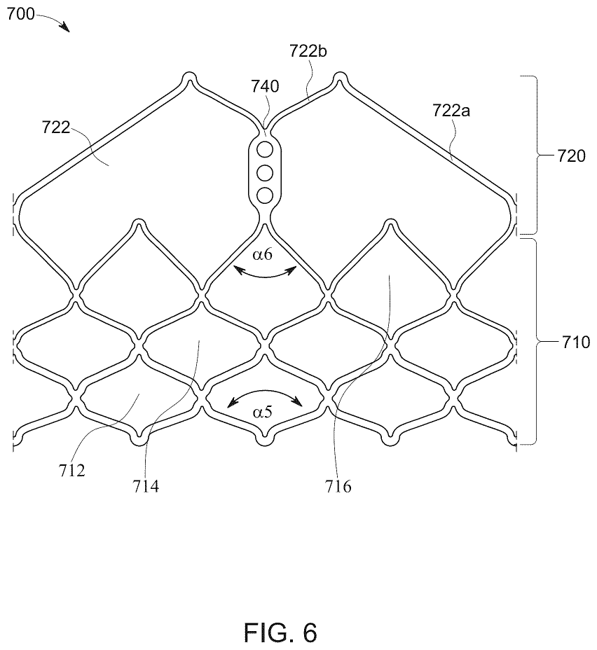

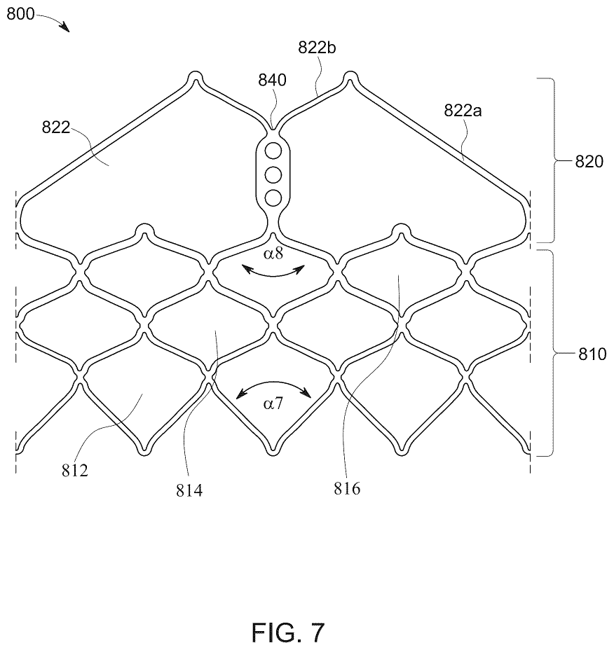

Although the stent 500 of FIG. 4A is described as having certain features to provide variable radial forces along the axial extent of the stent that include generally larger scale (or “macro”) designs, such as varying cell density, other smaller scale (or “micro”) designs may also be implemented to achieve varying radial force. For example, FIG. 5 shows a highly schematic drawing of a stent 600 (or a portion thereof, as with stent 500 of FIG. 4A), that includes certain features to adjust radial forces required to expand the stent 600 with a balloon or other expansion member. It should be understood that certain features, such as commissure attachment features, are not illustrated in FIG. 5, and FIG. 5 does not illustrate the entire circumferential or axial extent of the stent 600. Rather, FIG. 5 is intended to show how certain “micro” design variations may be implemented to adjust radial forces, for example including design features that may be implemented into stent 500 or other stent designs.

Referring to FIG. 5, which shows a portion of a stent 600 as if laid out flat, only two full rows of cells, including an inflow row of cells 612 and an intermediate row of cells 614, are shown. However, it should be understood that additional rows of cells could be provided to create a full stent 600, and as with stent 500, the portion of stent 600 shown in FIG. 5 may represent only a portion (e.g., only a third) of the entire circumferential extent of the stent 600. FIG. 5 better illustrates that the inflow cells 612 may include struts with varying width. For example, each inflow cell 612 may include two proximal struts 612a that each have a width that is greater than the width of two distal struts 612b of the cell 612. Each distal strut 612b of each inflow cell 612 may also form proximal struts of each intermediate cell 614. Each intermediate cell 614 may include two distal struts 614b that have a width that is smaller than that of struts 612b. With this configuration, the three rows of struts 612a, 612b, 614b that form the two rows of cells 612, 614 have decreasing widths in the inflow-to-outflow direction. In other words, the decreasing width of the struts provides a decreasing amount of radial force required to expand the stent 600 in the direction from the inflow-to-outflow end. Although not visible in FIG. 5, the thickness of struts 612a, 612b, 614b may be provided as decreasing thicknesses in the inflow-to-outflow direction. The decreasing thickness may be created in any suitable fashion. For example, the stent 600 may be laser cut from a tube having a single wall thickness, and after being cut, struts 612b can be shaved down to a smaller thickness than struts 612a, and struts 614b may be shaved down to a smaller thickness than struts 612b, etc. Including such a decreasing strut thickness in the inflow-to-outflow direction may similarly provide a decreasing amount of radial force required to expand the stent 600 in the direction from the inflow-to-outflow end.

The angles between struts forming the cells may also be manipulated to affect the amount of force required to expand the different sections of the stent 600. For example, the three rows of diamond-shaped cells 512, 514, 516 of the stent 500 of FIG. 4A are all substantially identical to each other in shape, and the two bottom struts of each cell are substantially mirror images of the two top struts of each cell. However, the same is not true of the stent 600 in FIG. 5. For example, the two struts 612a in each cell 612 are not mirror images of the other two struts 612b in each cell 612. Rather, each strut 612a has the same circumferential extent as each strut 612b, but the two struts 612a each have a significantly smaller axial extent than the two struts 612b. As a result of this, when the frame 600 is in the expanded state, the bottom two struts 612a form an angle α1 that is larger than the angle α2 formed between the top two struts 612b to form a kite-shaped cell 612. Similarly, referring to cells 614, each strut 612b has the same circumferential extent as each strut 614b, but the two struts 612b each have a significantly smaller axial extent than the two struts 614b. As a result of this, when the frame 600 is in the expanded state, the bottom two struts 612b of cell 614 form an angle α3 that is larger than the angle α4 formed between the top two struts 614b to form a kite-shaped cell 614. Another result of this configuration is that the sides of cells 612 form an angle β1 that is smaller than the angle β2 formed by the sides of cells 614.

In the simplest case, diamond cells with smaller opening angles (such as α4) will foreshorten less when expanded compared to diamond cells with larger opening angles (such as α1). Overall, the type of design shown in FIG. 5 will have less overall motion (e.g. distance D1 shown in FIG. 3B). Angle α1 is more open, and will thus foreshorten more during expansion. Strut cells with higher opening angle (e.g. α1) also have more radial strength than those with low opening angle e.g. (α4), all else being equal, since the struts with the larger opening angle are angled more in the circumferential direction in the expanded configuration. Thus, the combination of opening angle and strut width can have an effect on both the radial force and the amount of foreshortening of the diamond-shaped cells. The ratios of radial force across different struts in the axial direction can then have an impact on the way the valve frame 600 expands during balloon expansion. There are also factors from the balloon involved depending on its design that will cause the balloon to open in different ways. As a result, the interaction between the balloon and the relationship of axial forces on the valve frame may be designed to achieve a desired motion, with the goal generally being to achieve little or no axial motion on the inflow end of the frame 600 during expansion. Any one or more of these “micro” design features may be utilized, including for use in frame 500, to help achieve the results shown in FIGS. 4B-C in which the terminal inflow end of the stent does not move (or does not move significantly) in the axial direction between the collapsed condition (FIG. 4B) and the expanded condition (FIG. 4C).

Although the disclosure above is generally directed to using “macro” or “micro” design features of the stent of a prosthetic heart valve to help achieve desired foreshortening so that the inflow end of the prosthetic heart valve does not significantly shift in the axial direction during expansion, other features, including the interaction between the prosthetic heart valve and the balloon onto which it is crimped, may be utilized to help achieve similar results. For example, the outer surface of the balloon 380 may be treated to increase friction and/or decrease friction. For example, it may be desirable for the portion of the balloon 380 that will contact the inner surface of the prosthetic heart valve at the inflow end to have relatively high friction, and/or for the portion of the balloon 380 that will contact the inner surface of the prosthetic heart valve at the outflow end to have relatively low friction. Lower friction between the outflow end of the prosthetic heart valve and the balloon may make it easier for the cells of the stent to foreshorten (which requires axial movement relative to the balloon) during expansion. Low-friction surfaces may be created on the balloon by having a smooth surface, by using a low-friction material for that section of the balloon, and/or by including low-friction coatings on that section of the balloon. On the other hand, greater friction between the inflow end of the prosthetic heart valve and the balloon may make it more difficult for the cells to foreshorten during expansion. High-friction surfaces may be created on the balloon by using a roughened surface, high-friction materials (as a coextrusion or additional layer), coatings, plasma treatment, or other surface modifications for that section of the balloon. Similarly, the inner diameter of the prosthetic heart valve may be treated to create a desired amount of friction with the balloon by tailoring the tissue chemical processing or modifying the surface of the stent frame. The balloon/prosthetic heart valve assembly could alternatively or additionally be treated after crimping with chemical or thermal processes to improved adhesion.

Although FIG. 4A illustrates one example of a portion of a frame 500 suitable for use with a balloon-expandable prosthetic heart valve, various additional frames may be similarly suitable. The below disclosure describes various alternate embodiments compared to frame 500. It should be understood that features of frame embodiments may be combined with features of other frame embodiments, even if such a combination is not explicitly shown. For example, the outflow portions of a frame embodiment may be combined with the inflow portion of another frame embodiment, and similarly the CAFs of any embodiment may be replaced with the CAFs of any other embodiment. In addition, the “micro” design variations shown and described in connection with FIG. 5 may be applied to the other frame embodiments described below.

FIG. 6 shows an example of a portion of a frame 700 for use in a balloon-expandable prosthetic heart valve (e.g. a prosthetic aortic valve), as if cut longitudinally and laid on a table. As with frame 500, frame 700 represents only a third of the entire frame (if three prosthetic leaflets are to be used with frame 700), and the entire frame would consist of a total of three repeating patterns of the portion shown in FIG. 6, formed as a continuously cylindrical or tubular member. Similar to frame 500, the prosthetic heart valve that incorporates frame 700 may be formed from a plastically expandable material (e.g. stainless steel or cobalt chromium), for example by laser-cutting from a tube. Prosthetic leaflets and inner and/or outer sealing skirts or cuffs are not shown, but may be used with frame 700 to form the prosthetic heart valve similar to as described in connection with frame 500. The information in this paragraph pertains to the remaining frame portions shown in FIGS. 7-25, and is thus not repeated again for each embodiment in the interest of brevity.

As shown in FIG. 6, frame 700 may include an inflow section 710 and an outflow section 720. In the illustrated embodiment, the inflow section 710 may include a plurality of rows of generally diamond-shaped and/or kite-shaped cells. For example, the inflow section 710 may include an inflow-most row of diamond-shaped cells 712, an adjacent intermediate row of diamond-shaped cells 714, and an outflow-most row of diamond-shaped or kite-shaped cells 716. With this configuration, each cell in the intermediate row of cells 714 includes two struts that are shared with an adjacent cell in the inflow-most row of cells 712, and two struts that are shared with an adjacent cell in the outflow-most row of cells 716. It should be understood that although the term “outflow-most” is used in connection with the third row of cells 716, additional frame structure, described in more detail below, is still provided in the outflow direction relative to the outflow-most row of cells 716. An inflow apex of each cell in the first row of cells 712 may include an aperture or eyelet (similar to eyelet 513) formed therein, but in other embodiments, including the illustrated embodiment, the aperture or eyelet can be omitted. Although inflow section 710 may be generally similar in function to inflow section 510 of frame 500, a few differences may be provided (in addition to the omission of the inflow eyelets). For example, cells 712 and 714 may all include an opening angle α5 that is larger than that of cells 512 and 514. In the illustrated embodiment, opening angle α5 may be between about 130 degrees and about 150 degrees, including about 140 degrees. In the illustrated embodiment, cells 712, 714 may have bilateral symmetry in terms of the strut angles forming the cells. Cells 716 may include an outflow apex formed by struts that form an opening angle α6 that is smaller than opening angle α5. For example, opening angle α6 may be between about 80 degrees and about 100 degrees, including about 90 degrees. It should be understood that other opening angles than those explicitly mentioned above may be provided, with the general goal being that, by providing opening angle α5 larger than α6 (and thus struts that extend more in the circumferential direction for opening angle α5 compared to opening angle α6), cells 712 and 714 may provide greater radial force than cells 716, which may assist with better anchoring. In addition, in the illustrated embodiment, the four struts forming each cell 712 have a width that is larger than each of the four struts forming each cell 716, which may result in cells 712 being stiffer than cells 716. As used herein, the term opening angle generally refers to the angle between two struts of a cell when the frame is expanded to a typical operating condition (e.g. to the desired size expected to be used upon implantation into a native aortic valve).

Another difference between frame 700 compared to frame 500 is the CAF 740. Each CAF 740 may be formed integrally with the rest of the frame 700, and may generally define a vertical bar with one or more eyelets for accepting sutures therethrough. In the illustrated embodiment, each CAF 740 includes three eyelets in a single column, but other numbers of eyelets, and in different configurations (e.g. two-by-two grid) may be suitable. Each CAF 740 may extend axially from an outflow apex of one of the cells 716 in the outflow-most row of cells 716 of the inflow section 710.

Still referring to FIG. 6, the outflow section 720 of the stent 700 may include larger cells 722 that have shapes other than a regular polygon. For example, the lower part of the larger cells 722 may be defined by the two upper struts of a cell 716, and one upper strut of each of the two adjacent cells 716. In other words, the lower end of each larger cell 722 may be formed by a group of four consecutive upper struts of three circumferentially adjacent cells 716. The top of the larger cells 722 may be defined by two linking struts 722a, 722b. The first linking strut 722a may couple to a top or outflow apex of a cell 716 and extend upwards at an angle toward a CAF 740. The second linking strut 722b may extend from an end of the first linking strut 722a back downwardly at an angle and connect directly to the CAF 740 at a top or outflow side of the CAF 740. To the extent that the larger cells 722 include sides, a first side is defined by a portion of the CAF 740, and a second side is defined by the connection between first linking strut 722a and the corresponding upper strut of the cell 716 attached to the first linking strut 722a. Also, in the illustrated embodiment, the first linking struts 722a are provided with greater width than the second linking struts 722b, similar as described in connection with linking struts 522a-c. As with frame 500, the configuration shown and described in connection with frame 700 results in an outflow section 720 that has lower stiffness than the inflow section 710. Further, it should be understood that while many differences are described between frame 700 and frame 500, there are still many similarities, and relevant portions of the description of the function of frame 500 apply with substantially equal force to the function of frame 700.

FIG. 7 shows a portion of a frame 800 that includes alternate features compared to frame 700. It should be understood that only the differences are described here for brevity, and thus items that are not described should be understood to be the same or similar to corresponding elements of frame 700 (or frame 500). Similar to frame 700, frame 800 includes an inflow section 810 with three rows of cells, except the inflow-most row has a diamond-shape or kite-shape with a relatively small opening angle α7, which may be for example between about 90 degrees and about 110 degrees, including about 100 degrees. Intermediate row 814 and outflow row 816 may both be similar or identical to each other, having general diamond-shapes with an opening angle α8 which may be between about 120 degrees and about 140 degrees, including about 130 degrees. As with frame 700, the struts forming cells 812 may be wider than the struts forming cells 816. The outflow section 820 may be generally the same as the outflow section 720, including the general shape of cells 822 and CAFs 840. The first and second linking struts 822a, 822b may have a similar shape to struts 722a, 722b, although in thee illustrated embodiment the linking struts 822a, 822b have a width that is generally similar to the struts forming cells 812, so that the stiffness of the linking struts 822a, 822b is similar to the stiffness of the struts forming cells 812. It should be understood that particular features of frames 700 and 800 may be combined to form frame that is not shown, such as the combining the inflow section 710, 810 with the outflow section 720, 820 of the other frame.

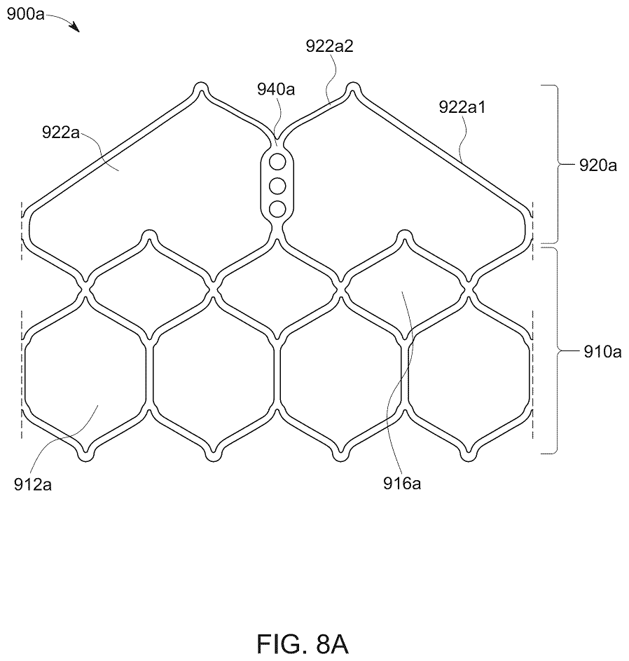

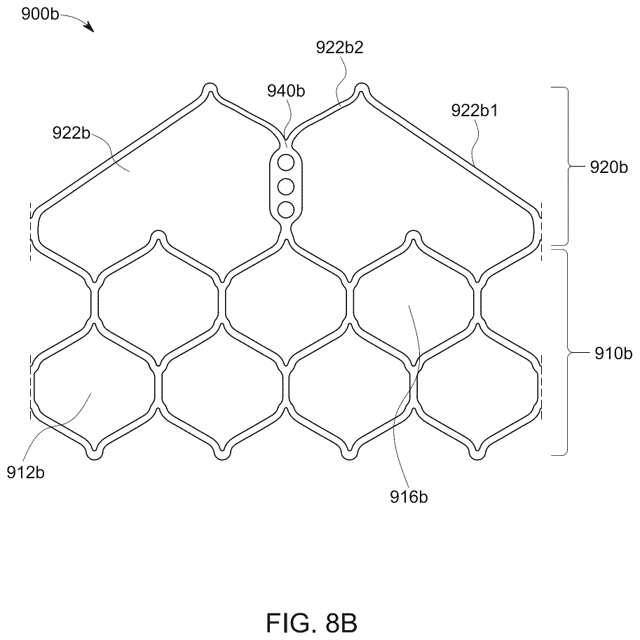

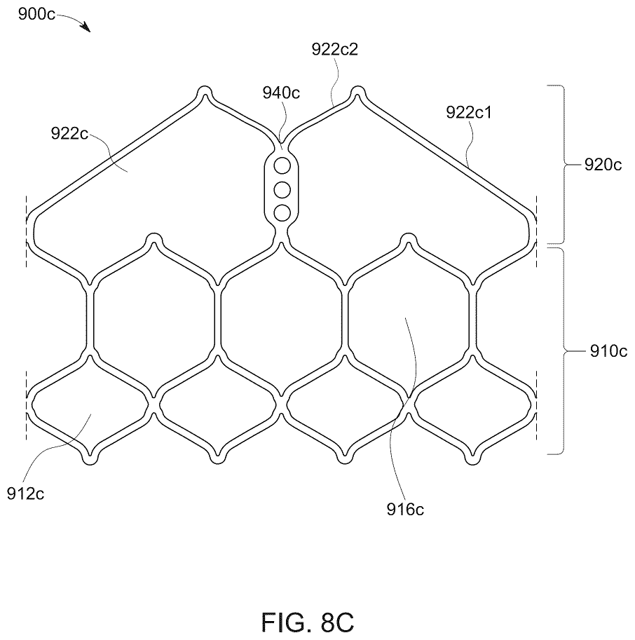

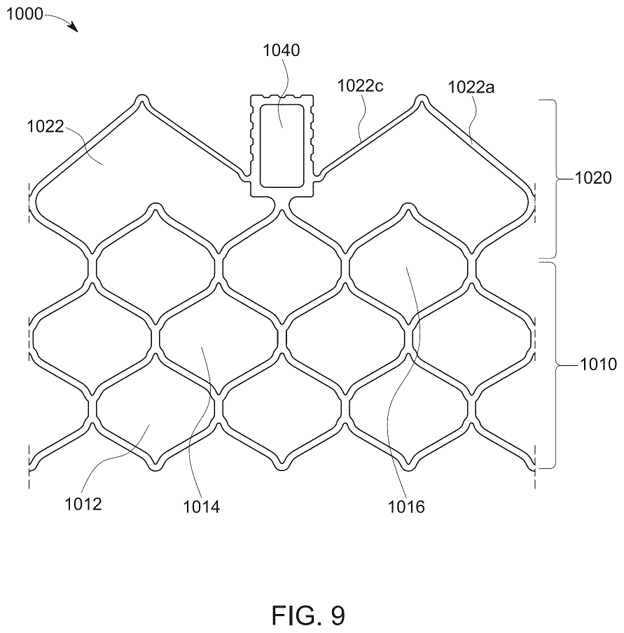

FIGS. 8A-C show different versions of a portion of a frame 900a-c that are generally similar to frames 700, 800. Thus, only the differences are described below, and non-described features should be understood to be similar or adjacent to the corresponding features of frames 700-800. Frames 900a-c each include an inflow section 910a-c and an outflow section 920a-c, respectively. The inflow section 910a-c of each frame 900a-c may include two rows of cells, including an inflow row 912a-c and an outflow row 916a-c. Each of the cells in the inflow sections 910a-c may be generally diamond-shaped, although some cells may have axial runners of different lengths to change the shapes of the specific cells. For example, cells 916a have axial runners with zero or near-zero length, whereas cells 912a have axial runners with larger length making cells 912a generally six-sided. Frame 900c has the opposite configuration compared to frame 900a, with cells 912c having axial runners with zero or near-zero length, and cells 916c having runners with larger length making cells 916c generally six-sided. Frame 900b, on the other hand, has an intermediate configuration in which cells 912b, 916c are substantially identical and each have axial runners that are longer than the runners of cells 912a, 916c but shorter than the runners of cells 912c, 916a. In some embodiments, the inflow sections 910a-c may all have the same axial length as each other, so that the entire frames 900a-c all have the same axial length or each other. This may be particularly true when the outflow sections 920a-c are identical to each other, as shown in FIGS. 8A-C. Outflow sections 920a-c may include larger cells 922a-c formed, in part, by first linking struts 922a1, 922b1, 922c1 and second linking struts 922a2, 922b2, and 922c2, with CAFs 940a-c. In the illustrated embodiments, outflow sections 920a-c are similar or identical to outflow section 820, although in other embodiments they may be similar or identical to any other outflow section described herein.

FIG. 9 illustrates another example of a portion of a frame 1000, which is generally similar to frame 500, except for certain differences that are described herein. For features not described in connection with frame 1000, it should be understood that those features may be similar or identical to frame 500. Similar to frame 500, frame 1000 includes an inflow section with three rows of cells, including inflow cells 1012, intermediate cells 1014, and outflow cells 1016. These cells may all be identical to one another and have a generally diamond shape. However, compared to cells 512, 514, 516, cells 1012, 1014, 1016 have slightly elongated axial runners (it should be understood that axial runners do not contribute to any axial foreshortening of the frame during expansion). Also, the inflow apices of cells 1012 omit any apertures or eyelets similar to eyelets 513. The outflow section 1020 is generally similar to outflow section 520, with two main differences. Large cells 1022 have a generally similar shape to cells 522, but include only two linking struts 1022a, 1022c instead of three linking struts 522a-c. Also, while CAFs 1040 are substantially identical to CAFs 540, linking struts 1022c connect to CAFs 1040 near a bottom (or inflow) side thereof, meaning that more axial length of the CAFs 1040 are unsupported by linking strut 1022c. This configuration may allow for more inward deflection of the CAFs 1040 during use (e.g. during ventricular diastole when the prosthetic leaflets are closed, and pressure is being applied on the prosthetic heart valve in the outflow-to-inflow direction).

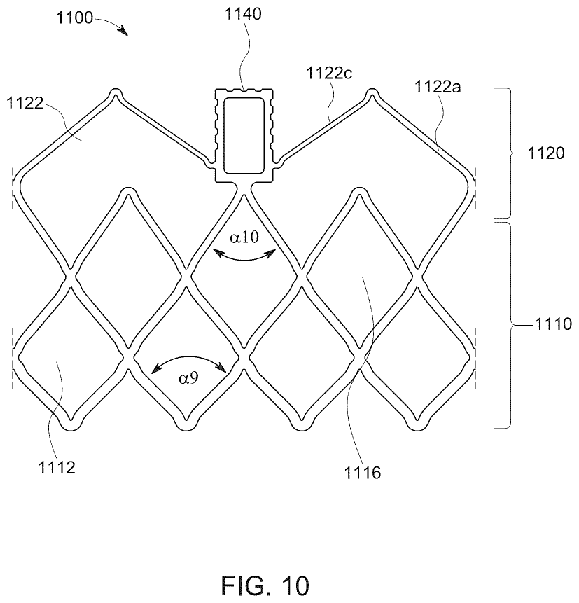

FIG. 10 illustrates another example of a portion of a frame 1100, which has features in common with frames 500 and 1000, except for certain differences that are described herein. For features not described in connection with frame 1100, it should be understood that those features may be similar or identical to frames 500 and/or 1000. Similar to frame 500, frame 1000 includes an inflow section, but with two (instead of three) rows of cells, including inflow cells 1112 and outflow cells 1116. Inflow cells 1112 and outflow cells 1116 may all be generally diamond-shaped cells that omit any axial runners (or at least omit axial runners of any significant length). Cells 1112 may have inflow struts that are wider than the outflow struts which are shared with cells 1116, and cells 1116 may have outflow struts that are thinner than the inflow struts which are shared with cells 1112. With this configuration, the struts forming the cells 1112, 1116 have a decreasing stiffness in the inflow-to-outflow direction. Further, cells 1112 may have an opening angle α9, which may be between about 80 degrees and about 100 degrees, including about 90 degrees, that is smaller than the opening angle α10 of cells 1116, which may be between about 60 degrees and about 80 degrees, including about 70 degrees. With this configuration, cells 1112 may have increased radial strength compared to cells 1116. The outflow section 1120 may be identical to outflow section 1020, including cells 1122 formed in part by two linking struts 1122a, 1122c, with linking strut 1122c connecting to CAF 1140 near a bottom or inflow side of the CAF 1140.

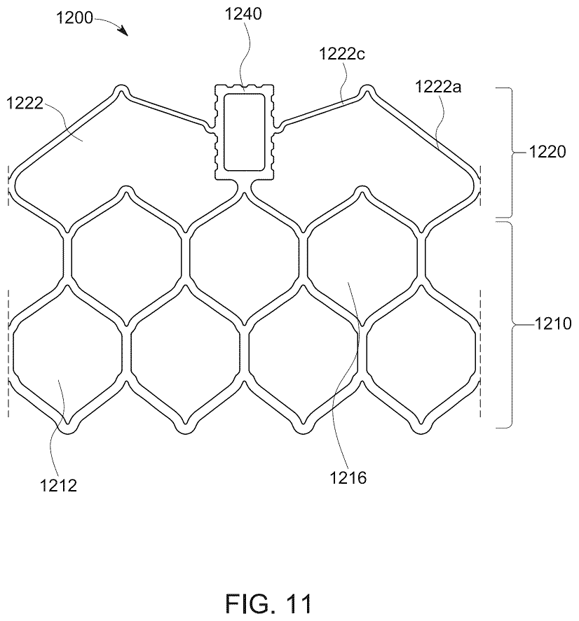

FIG. 11 illustrates another example of a portion of a frame 1200, which has features in common with frame 1100, except for certain differences that are described herein. For features not described in connection with frame 1200, it should be understood that those features may be similar or identical to frame 1100. Similar to frame 1100, frame 1200 includes an inflow section with two rows of cells, including inflow cells 1212 and outflow cells 1216. However, unlike cells 1112, 1116, cells 1212, 1216 each include axial runners having a substantial length so that cells 1212, 1216 are generally six-sided. As with inflow section 1110, the struts of inflow section 1210 may be widest at the inflow struts of cells 1212, intermediate width at the struts shared between cells 1212, 1216, and thinner at the outflow struts of cells 1216. The outflow section 1220 may be generally similar to outflow section 1120, including large cells 1222 formed in part by two linking struts 1222a, 1222c. However, linking struts 1222c are shown as connecting to an axial mid-point of CAFs 1240, which may provide additional stability to the CAFs 1240 compared to the configuration of frame 1100.

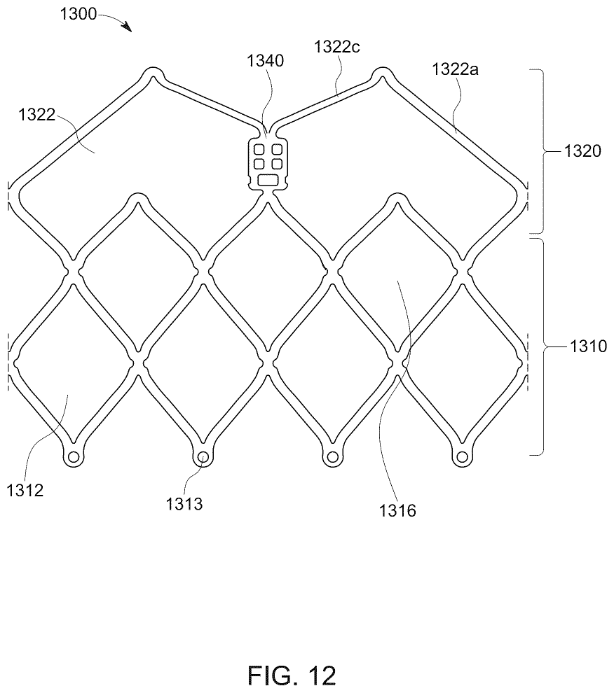

FIG. 12 illustrates another example of a portion of a frame 1300, which has features in common with frame 1100, except for certain differences that are described herein. For features not described in connection with frame 1300, it should be understood that those features may be similar or identical to frame 1100. Similar to frame 1100, frame 1300 includes an inflow section with two rows of cells, including inflow cells 1312 and outflow cells 1316. Cells 1312 and 1316 may both be generally diamond-shaped and may have uniform strut widths, with similar or identical opening angles. The inflow section 1310 is similar to inflow section 510 in that the inflow struts of inflow cells 1312 include eyelets 1313 at inflow apices to assist with suturing material (e.g. inner and/or outer skirt/cuff material and/or leaflet material) to the frame 1300. The outflow section 1320 may be generally similar to outflow section 820 in that the outflow section 1320 includes large cells 1322 formed in part by two linking struts 1322a, 1322c, with first linking struts 1322a being wider than second linking struts 1322c, and second linking struts 1322c connecting to a CAF 1340 near a top thereof. However, CAFs 1340 are different than other CAFs described and shown above, with the CAFs 1340 having a generally rectangular body with a two-by-two arrangement of small eyelets with a single elongated eyelet below the two-by-two arrangement of small eyelets. As with other embodiments described herein, the connection of the second linking strut 1322c to the top of the CAF 1340 may provide more stability to the CAF 1340 compared to mid-point or bottom connections to the CAF.

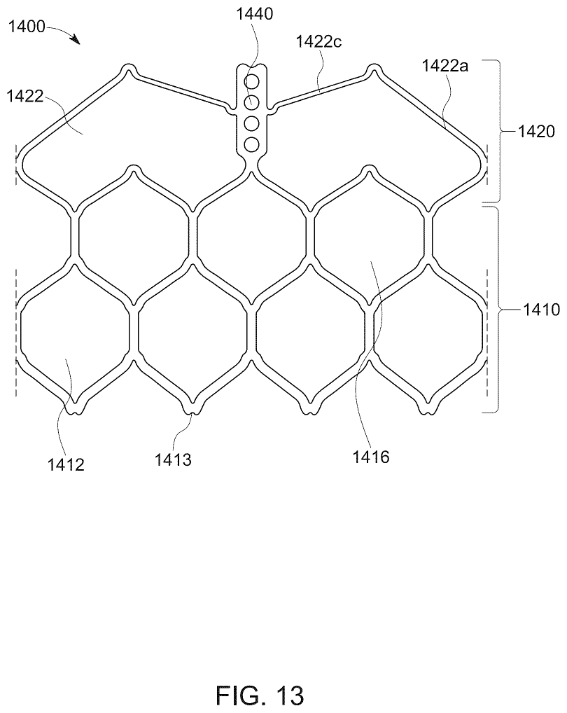

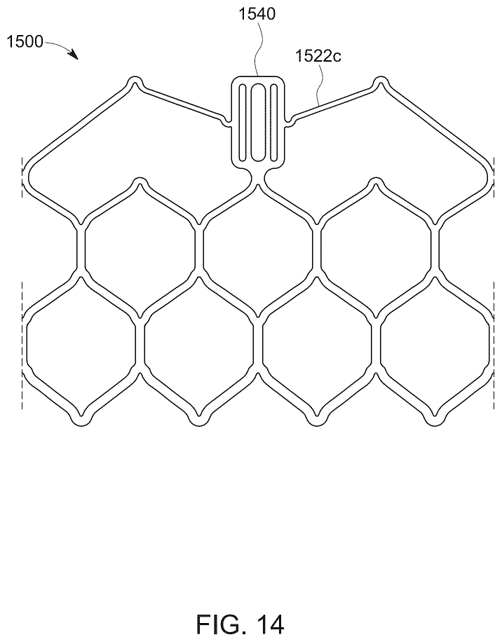

FIG. 13 illustrates another example of a portion of a frame 1400, which has features in common with frame 1200, except for certain differences that are described herein. For features not described in connection with frame 1400, it should be understood that those features may be similar or identical to frame 1200. Similar to frame 1200, frame 1400 includes an inflow section 1410 with two rows of cells, including inflow cells 1412 and outflow cells 1416. However, unlike cells 1212, 1216, cells 1412, 1416 are each shown with struts having equal widths to each other. The outflow section 1420 may be generally similar to outflow section 1220, including large cells 1422 formed in part by two linking struts 1422a, 1422c. However, although linking struts 1422c are shown as connecting to an axial mid-point of CAFs 1440, CAFs 1440 have a different form than CAFs 1240. CAFs 1440 are formed as generally axially-extending bars (similar to CAFs 840), which are shown with a single column of four eyelets, although more or fewer eyelets may be included in each CAF 1440, either in a single column or a different arrangement. The inflow apices of cells 1412 may include a notch 1413, which may serve a similar purpose as eyelets 513. In other words, sutures may wrap around notches 1413 to help secure the position of the sutures relative to the frame 1400. The outflow end of CAFs 1440 may include a similar notch for helping to secure sutures.

FIG. 14 illustrates another example of a portion of a frame 1500, which is identical to frame 1400 except of the CAF 1540. All other features of frame 1500 are thus not described. While CAF 1540 connects to second linking strut 1522c near an axial midpoint of the CAF 1540, the CAF 1540 has a configuration not shown or described above. For example, each CAF 1540 may be generally rectangular, including two horizontal or circumferential struts defining the top and bottom of the CAF 1540, and four axial struts or posts connecting the horizontal struts. The terminal ends of the horizontal struts may be coupled together by a pair of axial posts to define a generally rectangular shape. Two additional axial struts or posts may connect the horizontal struts at locations inward of the outer-most struts. With this configuration, three axially-elongated openings are formed by the CAFs 1540. In some embodiments, the tabs or side edges of two adjacent prosthetic leaflets may come together and extend through the central opening of the CAF 1540, with the tabs or side edges then laying generally flat against the outer surface of the CAF 1540. However, it should be understood that other particular configurations for attaching the prosthetic leaflet commissures to the CAFs 1540 may be suitable.

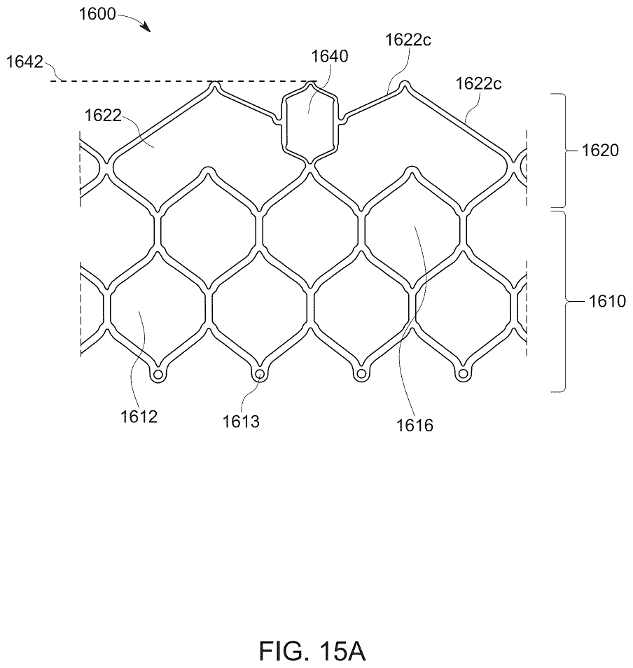

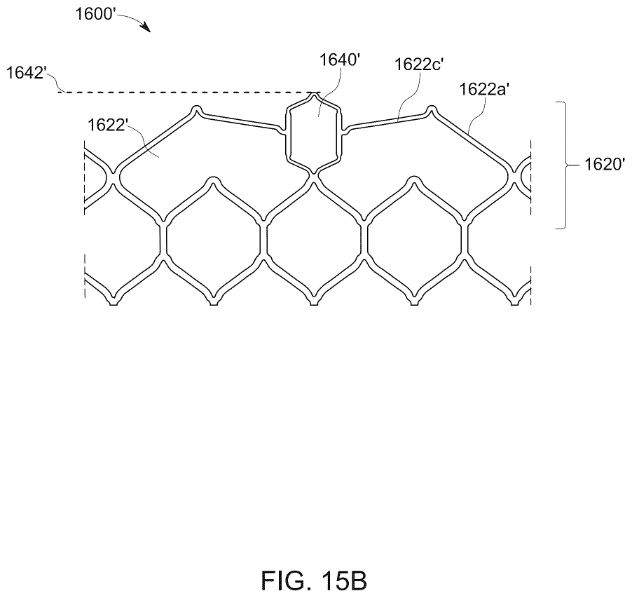

FIG. 15A illustrates another example of a portion of a frame 1600, which has features in common with frame 1200, except for certain differences that are described herein. For features not described in connection with frame 1600, it should be understood that those features may be similar or identical to frame 1200. Similar to frame 1200, frame 1600 includes an inflow section 1610 with two rows of cells, including inflow cells 1612 and outflow cells 1616. However, unlike cells 1212, 1216, cells 1612, 1616 are each shown with struts having equal widths to each other. Also, similar to frame 500, the inflow apices of cells 1612 include eyelets 1613. The outflow section 1620 may be generally similar to outflow section 1220, including large cells 1622 formed in part by two linking struts 1622a, 1622c. However, although linking struts 1622c are shown as connecting to an axial mid-point of CAFs 1640, CAFs 1640 have a different form than CAFs 1240. CAFs 1640 have a generally six-sided shape with two axial struts (one each connecting to a linking strut 1622c), and two pairs of struts forming apices, each strut apex coupling the two axial struts (one inflow strut apex, one outflow strut apex). With this configuration, CAF 1640 may be collapsible. In other words, as a prosthetic heart valve that incorporates frame 1600 is crimped to a smaller size (e.g. onto a delivery device for transcatheter delivery), the axial struts of CAF 1640 may move closer to each other as the two strut apices become more acutely angled. The collapsibility of CAF 1640 may enable a smaller overall crimped diameter for the prosthetic heart valve that incorporates frame 1600, and may thus allow better access to the patient. Upon expansion (e.g. by balloon inflation) of frame 1600, the CAF 1640 is re-expanded and provides a large open space for attachment and flexing of “soft” components (e.g. fabric and/or tissue cuffs and/or prosthetic leaflets) that are attached directly or indirectly to the CAF 1640.

FIG. 15B illustrates the outflow portion 1620′ of a portion of frame 1600′, which has a single modification compared to frame 1600, and thus only the modification is described, with the remainder of frame 1600′ being identical to frame 1600. Although frame 1600′ includes large cells 1622′ formed in part by two linking struts 1622a′, 1622c′, and although the second linking struts 1622c′ also couple to CAF 1640′ near an axial midpoint thereof, the configuration of linking struts 1622a′, 1622c′ is different from that of linking struts 1622a, 1622c. The difference is best shown by comparing dashed line 1642 in FIG. 15A to dashed line 1642′ in FIG. 15B. When the frame 1600 is in an expanded condition, the junction of linking struts 1622a, 1622c is at about the same axial height (or in circumferential alignment) as the outflow apex of CAF 1640. On the other and, in FIG. 15B, when the frame 1600′ is in an expanded condition, the junction of linking struts 1622a′, 1622c′ is axially below (in the inflow direction) the outflow apex of CAF 1640′. By lowering the linking strut 1622c′ (compared to linking strut 1622c) so that the linking strut 1622c′ is more horizontally or circumferentially oriented (compared to linking strut 1622), as the prosthetic heart valve that incorporates frame 1600′ is forced to expand, the linking strut 1622c′ is better situated to “pull” the CAF 1640′ into the open or expanded condition.

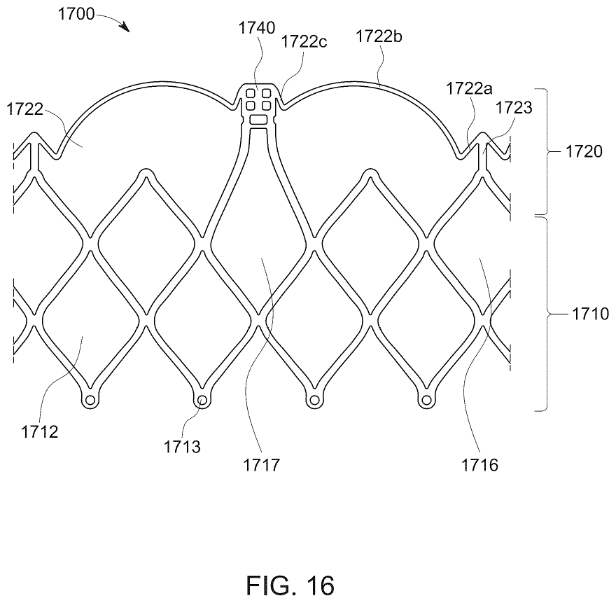

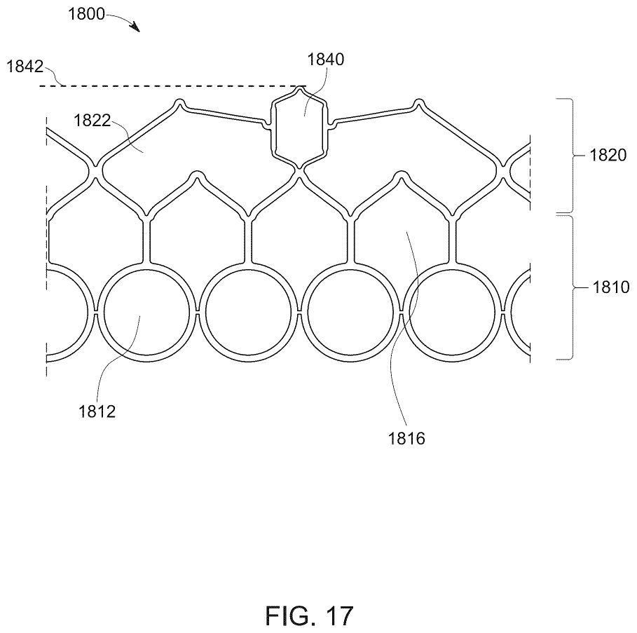

FIG. 16 illustrates another example of a portion of a frame 1700, which has features in common with frame 1300, except for certain differences that are described herein. For features not described in connection with frame 1700, it should be understood that those features may be similar or identical to frame 1300. Similar to frame 1300, frame 1700 includes an inflow section 1710 with two rows of cells, including inflow cells 1712 and outflow cells 1716, which may all be formed of struts having similar or identical widths. Inflow cells 1712 may all be generally diamond-shaped and similar or identical to each other cell in the same row, and the inflow apex of some or all inflow cells 1712 may include eyelets 1713 similar to eyelets 513. The outflow row of cells 1716 may also be generally diamond-shaped, but selected cells in the outflow row may be formed as enlarged cells 1717. In the illustrated embodiment, one enlarged cell 1717 is provided in the outflow row of cells 1716 for each CAF 1740 (e.g. a total of three enlarged cells 1717 and a total of three CAFs 1740 for a three-leaflet prosthetic valve). CAF 1740 may be substantially similar or identical to CAF 1340, but in the illustrated embodiment, instead of the CAF 1340 being located above an outflow apex of cell 1316, the bottom (or inflow side) of CAF 1740 is connected two struts forming the outflow end of enlarged cell 1717. In other words, the outflow end of the enlarged cell 1717 does not need to come to an apex, but rather the two outflow struts of enlarged cells 1717 may directly transition into two side edges of the CAF 1740. In addition to the inclusion of elongated cells 1717, the main difference between frame 1700 and frame 1300 is the enlarged cells 1722. Each enlarge cell 1722 in the outflow section 1720 may be formed, in part, by three linking struts 1722a-c. In the illustrated embodiment, a first linking strut 1722a is connected to an axial runner 1723 that extends in the outflow direction from a selected cell 1716 in the outflow row. The first linking strut 1722a may be short and be angled toward the inflow side of the frame 1700. A second linking strut 1722b may extend most of the distance between the axial runner 1723 and CAF 1740. In the illustrated example, the second linking strut 1722b is curved (e.g. shaped as a portion of a circle) with the convex side of the curvature pointing generally in the outflow direction and the concave side of the curvature pointing generally in the inflow direction. The two ends of the curved second linking strut 1722b may be coupled to ends of the first linking strut 1722a and the third linking strut 1722c. The third linking strut 1722c may extend upwardly or in the outflow direction and connect at or near the top (or outflow end) of the CAF 1740. By using an arched strut design (referring mainly to second linking strut 1722b), stresses may be more evenly distributed across the length of the arched strut during collapse and expansion of the frame 1700, compared to designs with only linear lining struts. This elastic energy may help to minimize crimp strains, and may be used to help center the CAF 1740 by flexing against each other on an expanded frame 1700.

FIG. 17 illustrates another example of a portion of a frame 1800, which has features in common with frames 1600, 1600′, except for certain differences that are described herein. For features not described in connection with frame 1800, it should be understood that those features may be similar or identical to frame 1600 or 1600′. The outflow section 1820 of frame 1800 is identical to the outflow section 1620′ of frame 1600, including the shape of the large cells 1822 and the position of the linking struts (see dashed line 1842) relative to the CAF 1840. The inflow section 1810, however, is different than any inflow section described above. In particular, inflow section 1810 includes inflow cells 1812 and outflow cells 1816. Inflow cells 1812 generally take the shape of complete circles, with each adjacent circular cell 1812 coupled via a short horizontal linking strut (shown but not separately labeled in FIG. 17). In the illustrated embodiment, four circular inflow cells 1812 are provided for each section of the frame 1800 (e.g. a total of twelve circular inflow cells 1812 in a three-leaflet prosthetic heart valve). The outflow cells 1816 may be partially formed as diamond-shaped cells. For example, the outflow end of each outflow cell 1816 may be formed as a diamond-shaped cell, with elongated axial runners extending from the outflow strut apex of each cell 1816. The elongated axial runners may be directly connected to the top (or outflow end) of one of the circular inflow cells 1812. With this frame design that includes an alternating cell structure with half-diamond-shaped cells 1816 and full circular cells 1812, the frame may be particularly suited to preserving uniformity of shape during collapse and expansion, while using the distributed stress/strain on the circular arc(s) of cells 1812 to control stent frame recoil.

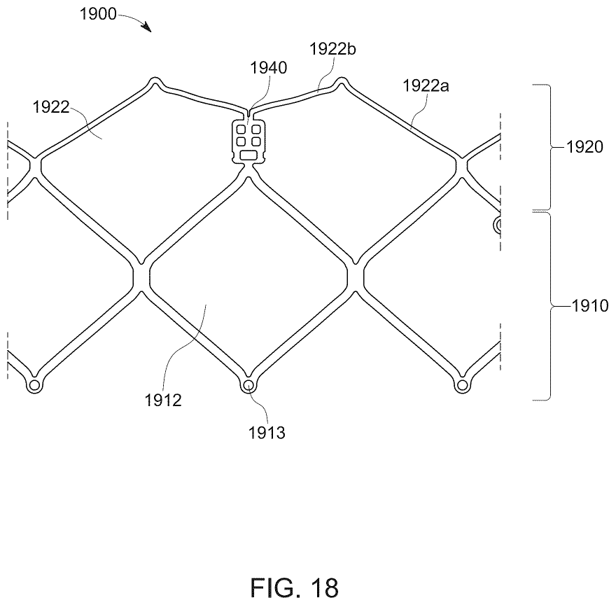

FIG. 18 illustrates another example of a portion of a frame 1900, which has features in common with frame 13, except for certain differences that are described herein. For features not described in connection with frame 1900, it should be understood that those features may be similar or identical to frame 1300. The inflow section 1910 of frame 1900 is different than all other embodiments disclosed above in the sense that the inflow section 1910 is formed of only a single row of cells 1912, which may be significantly larger than the inflow cells of other embodiments described herein. For example, as shown in FIG. 18, cells 1912 may be generally diamond-shaped and may include only three cells per CAF 1940 (e.g. a total of twelve cells 1912 for an embodiment with three prosthetic leaflets). The axial height of inflow section 1910 may be similar to the axial heights of other inflow sections described herein, despite the fact that the inflow section 1910 includes only a single row of cells 1912. In the illustrated embodiment, the inflow apices of cells 1912 include an eyelet 1913 similar to eyelets 513. Due in part to the larger size of cells 1912, none of the cells 1912 have an outflow apex that is not directly coupled to a strut of a cell 1922 or a CAF 1940. The outflow section 1920 may be generally similar to outflow section 1320, including the fact that the outflow section 1920 may include large cells 1922 formed in part by linking struts 1922a, 1922b. Each linking strut 1922a may directly connect to an outflow apex of one of the large inflow cells 1912, and each linking strut 1922b may couple to a top or outflow side of CAF 1940, which may be similar or identical to CAF 1340. The large design of cells 1912 may help enhance coronary artery access. For example, after implantation of a prosthetic heart valve that incorporates frame 1900, it would be desirable for structure of the prosthetic heart valve to avoid obstructing potential future access to the coronary arteries. Due to the design of frame 1900, the large cells 1922 have a greater amount of open area compared to other large cells described herein. For example, comparing large cells 1922 to large cells 1322, it can be seen that because there is greater circumferential spacing between the outflow apices of adjacent inflow cells 1912 (compared to spacing between cells 1316), the large cells 1922 have larger areas. If one of the large cells 1922 is aligned with a coronary artery after implantation, it may be easier to pass a device (e.g. an interventional catheter) through the large cell 1922 and into the coronary artery, for example to place a coronary stent. “Soft” components (e.g. inner or outer fabric or tissue cuffs, or fabric or tissue prosthetic leaflets) of the prosthetic heart valve that incorporates frame 1900 may be attached to the large inflow cells 1912 at different desired tensions to produce different results. For example, these soft components may be tightly attached to create a taut fabric (or tissue) or may be more loosely attached to create a loose fabric (or tissue) connection. In some instances, the attachment may be tighter or looser depending on the particular cell. For example, an inner cuff may be tightly coupled to the cells 1912 that do not directly axially align with a CAF 1940, but loosely coupled to the cells 1912 that do directly axially align with a CAF 1940. With this configuration, the looser attachment areas may create extra flexing abilities for the leaflet attachment, which may help to reduce stresses that are applied directly to the tissue leaflet connections.

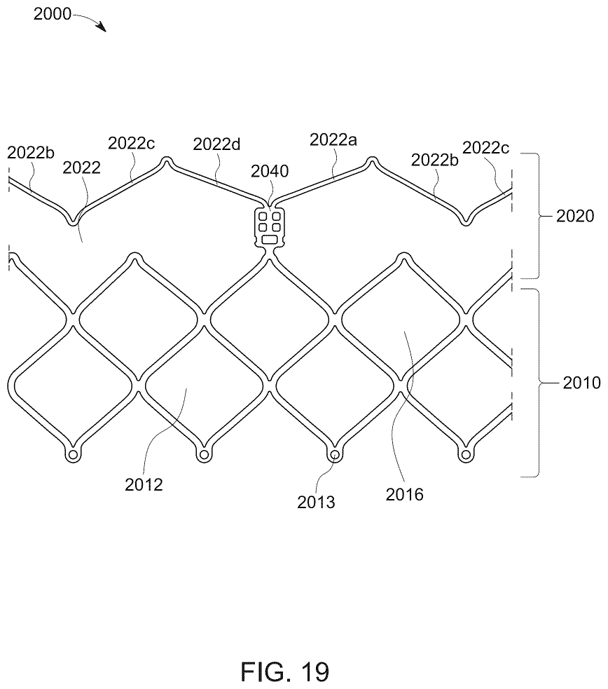

FIG. 19 illustrates another example of a portion of a frame 2000, which has features in common with frame 1300, except for certain differences that are described herein. For features not described in connection with frame 2000, it should be understood that those features may be similar or identical to frame 1300. Similar to frame 1300, frame 2000 includes an inflow section 2010 with two rows of cells, including inflow cells 2012 and outflow cells 2016, which may all be formed of struts having similar or identical widths. Inflow cells 2012 and outflow cells 2016 may all be generally diamond-shaped and similar or identical to each other cell in the same row, and the inflow apex of some or all inflow cells 2012 may include eyelets 2013 similar to eyelets 513. The outflow section 2020 may include CAFs 2040 that are similar or identical to CAFs 1340. The main difference between frame 1300 and frame 2000 is the configuration of large cells 2022 in the outflow section 2020. In particular, in the embodiment shown in FIG. 19, four linking struts 2022a-d are provided in a zig-zag fashion between circumferentially adjacent CAFs 2040, with the linking struts 2022a-d not directly connecting to cells 2016. For example, first linking strut 2022a may be directly attached to CAF 2040, for example at or near a top or outflow end thereof. First linking strut 2022a may connect to second linking strut 2022b, with the connection forming an apex pointing in the outflow direction. Second linking strut 2022b may connect to third linking strut 2022c, with the connection forming an apex pointing in the inflow direction. Third linking strut 2022c may connect to fourth linking strut 2022d, with the connection forming an apex pointing in the outflow direction. Finally, fourth linking strut 2022d may attach directly to one of the CAFs 2040, for example at or near a top or outflow end thereof. With this configuration, the linking struts 2022a-d are disconnected from most of the structure of frame 2000, except for the CAFs 2040. This configuration may help to provide a relatively even collapsing and expansion of the large cells 2022, while simultaneously acting as a thin top or outflow ring of the frame 2000. The function as a thin outflow ring may help control (e.g. minimize) the degree of flaring of the outflow end of the frame 2000 when the prosthetic heart valve is expanded, while also helping to ensure the native aortic valve leaflets are pinned back and prevented from interfering with the operation of the prosthetic heart valve that incorporates frame 2000.

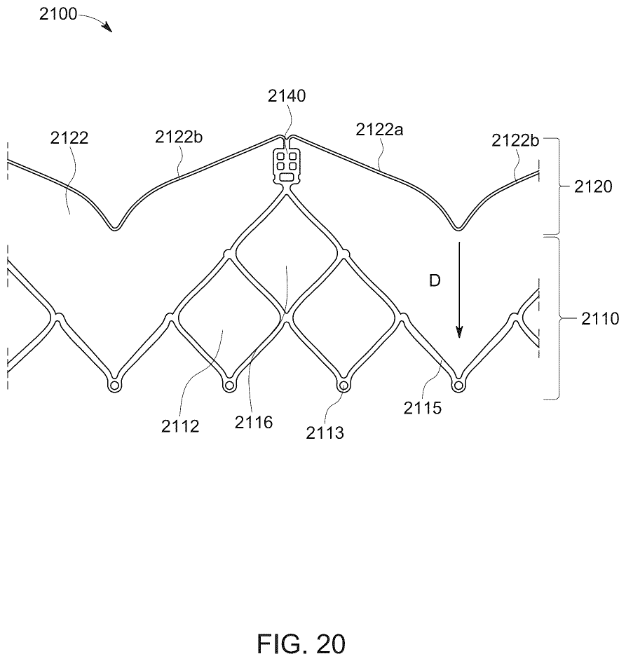

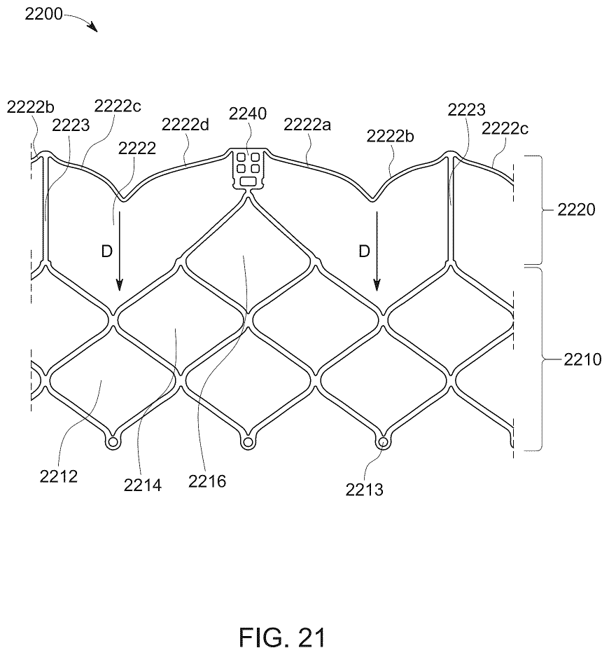

FIG. 20 illustrates another example of a portion of a frame 2100. Frame 2100 illustrates a little more than a third of the entire frame 2100 (assuming the frame 2100 is to be used with a three-leaflet prosthetic heart valve). Frame 2100 may include an inflow section 2110 and an outflow section 2120. The inflow section 2110 may have an inflow-most row of cells 2112, although the cells 2112 are not continuous around the circumference of the row. For example, FIG. 20 illustrates that the inflow-most row of cells 2112 may include a pair of diamond-shaped cells on either circumferential side of the CAF 2140, and a gap formed by large cells 2112 between each pair of adjacent cells 2112. In a three-leaflet embodiment, frame 2100 may include a total of six inflow-most cells 2112. The inflow section 2110 may also include an outflow row of cells 2116. As with cells 2112, cells 2116 are not continuously distributed around the circumference of the frame. Rather, a single diamond-shaped cell is positioned directly underneath each CAF 2140, and is formed of struts that form part of pairs of inflow cells 2112.

The inflow cell 2112 of one pair of inflow cells may couple to the inflow cell 2112 of an adjacent pair of inflow cells 2112 by two struts 2115 that form a “V”-shape with an apex pointing in the inflow direction. In the illustrated embodiment, struts 2115 have a width that is larger than the widths of the struts that form cells 2112 and cells 2116. The outflow section 2120 may include very large cells 2122 that are not isolated to the outflow section 2120. In the illustrated example, each adjacent pair of CAFs 2140 is connected by two linking struts 2122a, 2122b. Each linking strut 2122a, 2122b has a first end that couples to a CAF 2140 at or near a top or outflow edge thereof, and opposite ends that connect to each other. In the illustrated example, the connection between linking struts 2122a, 2122b forms an apex that points toward the inflow end of the frame 2100. In some embodiments, including the illustrated embodiment, the linking struts may flare toward the outflow end of the frame 2100 near their connection point, forming a pronounced “V”-shaped apex, which may have an apex angle similar to that formed by struts 2115. With this configuration, the very large cells 2122 are bounded by the linking struts 2122a, 2122b at an outflow end, CAFs 2140 at a side edge, and struts of cells 2116, 2112 and struts 2115. The design illustrated in FIG. 20 may provide for very small amounts of foreshortening during expansion of the frame 2100. The linking struts 2122a, 2122b provide support to the outflow end of the frame 2100 (including providing support to the CAFs 2140), but upon collapsing the frame, the V-shaped connection between linking struts 2122a, 2122b may shift downward in direction D and eventually nest within the collapsed V-shaped struts 2115. Other features of the frame 2100, including methods of creation and materials that can form the frame 2100, as well as features of a prosthetic heart valve that incorporate frame 2100, may be similar or the same as other embodiments described here.

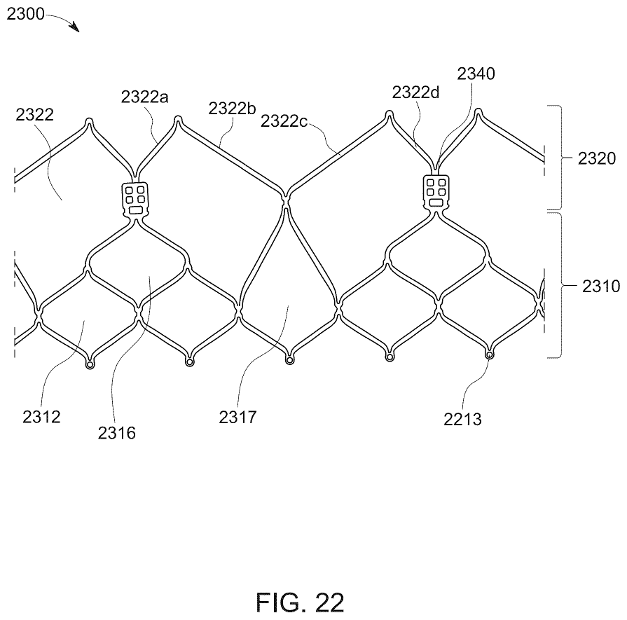

FIG. 21 illustrates another example of a portion of a frame 2200. Frame 2200 illustrates a little more than a third of the entire frame 2200 (assuming the frame 2200 is to be used with a three-leaflet prosthetic heart valve). Frame 2200 may include an inflow section 2210 and an outflow section 2220. The inflow section 2210 may have an inflow-most row of cells 2212 and an adjacent intermediate row of cells 2214. In the illustrated embodiment, cells 2212 and 2214 are all substantially diamond-shaped and provided in complete rows (e.g. 9 continuous diamond-shaped cells in sequence in each row). The inflow cells 2112 may include inflow apices that define eyelets 2213, which may be similar to eyelets 513. The inflow section 2210 may also include an outflow-most row of cells 2216, which may also be generally diamond-shaped, although the cells 2216 are not continuous around the circumference of the row. For example, FIG. 21 illustrates that the outflow-most row of cells 2216 may include a single diamond-shaped cell 2216 directly under each CAF 2240 (which may be similar or identical to CAFs 1340). In a three-leaflet embodiment, frame 2200 may include a total of three outflow-most cells 2216.

In the illustrated example, four linking struts 2222a-d are provided in a zig-zag fashion between circumferentially adjacent CAFs 2240. For example, first linking strut 2222a may be directly attached to CAF 2240, for example at or near a top or outflow end thereof. First linking strut 2222a may connect to second linking strut 2222b, with the connection forming an apex pointing in the inflow direction. Second linking strut 2222b may connect to third linking strut 2222c, with the connection forming an apex pointing in the outflow direction. Third linking strut 2222c may connect to fourth linking strut 2222d, with the connection forming an apex pointing in the inflow direction. Finally, fourth linking strut 2222d may attach directly to one of the CAFs 2240, for example at or near a top or outflow end thereof. At the connection between the second linking strut 2222b and the third linking strut 2222c, an axial linking strut 2223 may connect to span between the connection between struts 2222b, 2222c to the outflow apex of one of the intermediate cells 2214. In the illustrated embodiment, each axial linking strut 2223 is positioned about midway between each adjacent pair of CAFs 2240. Similar to frame 2100, as frame 2300 collapses, the inflow-pointing apices between struts 2222a, 2222b and between struts 2222c, 2222d move downwardly in direction D and substantially nest within or between struts of cells in the inflow section 2210.

The embodiment shown in FIG. 21 is another design that helps to minimize foreshortening, similar to frame 2100. Again, the top row of large cells 2222 in the outflow section 2220 is designed to move or shift down towards the inflow section 2210 upon collapsing, and thus will foreshorten in the middle of the frame structure and not increase the overall length. Other features of the frame 2200, including methods of creation and materials that can form the frame 2200, as well as features of a prosthetic heart valve that incorporate frame 2200, may be similar or the same as other embodiments described here.

FIG. 22 illustrates another example of a portion of a frame 2300. FIG. 22 illustrates nearly two-thirds of the entire frame 2300 (assuming the frame 2300 is to be used with a three-leaflet prosthetic heart valve). Frame 2300 may include an inflow section 2310 and an outflow section 2320. The inflow section 2310 may have an inflow-most row of cells 2312 and an adjacent outflow row of cells 2316. In the illustrated embodiment, cells 2312 and 2316 are all substantially diamond-shaped, but are not provided in complete rows. Rather, in the illustrated embodiment, two diamond-shaped inflow rows 2312 are provided in pairs on either circumferential side of each CAF 2340 (which may be similar or identical to CAFs 1340), for a total of six cells 2312 in a three-leaflet valve. Outflow cells 2316 are provided, in the illustrated embodiment, directly below each CAF 2340, for a total of three diamond-shaped cells 2316 in a three-leaflet valve. Between each pair of side-by-side inflow cells 2312, an elongated cell 2317 may be provided. Elongated cells 2317 may have a diamond-shape or a kite-shape, with each single elongated cell 2317 extending the entire axial distance of the inflow section 2310. In the illustrated embodiment, the inflow apices of the inflow cells 2312, as well as the elongated cells 2317, may include eyelets 2213 similar or identical to eyelets 513.

In the illustrated example, four linking struts 2322a-d are provided in a zig-zag fashion between circumferentially adjacent CAFs 2340. For example, first linking strut 2322a may be directly attached to a CAF 2340, for example at or near a top or outflow end thereof. First linking strut 2322a may connect to second linking strut 2322b, with the connection forming an apex pointing in the outflow direction. Second linking strut 2322b may connect to third linking strut 2322c, with the connection forming an apex pointing in the inflow direction. Third linking strut 2322c may connect to fourth linking strut 2322d, with the connection forming an apex pointing in the outflow direction. Finally, fourth linking strut 2322d may attach directly to one of the CAFs 2340, for example at or near a top or outflow end thereof. The connection between the second linking strut 2322b and the third linking strut 2322c may also serve as the connection to the outflow apex of the elongated cell 2317.

The embodiment shown in FIG. 22 is a has a non-symmetric cell design of alternating tall and short rows of cells, alternating numbers of rows, and alternating strut lengths to create an overall linkage system that creates open space for coronary access while also creating structure for leaflet attachment to the stent frame. Other features of the frame 2300, including methods of creation and materials that can form the frame 2300, as well as features of a prosthetic heart valve that incorporate frame 2300, may be similar or the same as other embodiments described here.

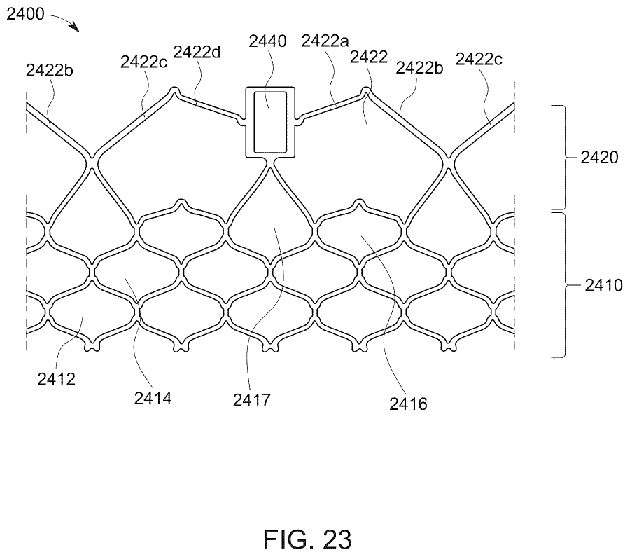

FIG. 23 illustrates another example of a portion of a frame 2400, with FIG. 23 showing slightly over one-third of the frame 2400 (assuming the frame is being used with three prosthetic leaflets). Frame 2400 may include an inflow section 2410 and an outflow section 2420. The inflow section 2410 may have an inflow-most row of cells 2412, an adjacent intermediate row of cells 2414, and an outflow row of cells 2416. In the illustrated embodiment, cells 2412, 2414, and 2416 are all substantially diamond-shaped. Rows 2412 and 2414 may be provided in complete rows of identical cells, whereas row 2416 may include elongated cells 2417 (which may have a diamond-shape or kite-shape) that alternate with diamond-shaped cells 2416. Elongated outflow cells 2417 are provided, in the illustrated embodiment, directly below each CAF 2440, as well as directly below the connection of linking struts 2422b, 2422c (described in greater detail below), for a total of six diamond-shaped cells 2416 and six elongated outflow cells 2417 in a three-leaflet valve. Each elongated cell 2417 may extend an axial distance toward the outflow end that is greater than the axial extent of the diamond-shaped cells 2416. In the illustrated embodiment, the inflow apices of the inflow cells 2412 may include notches 2413, which may be similar or identical to notches 1413.

In the illustrated example, four linking struts 2422a-d are provided in a zig-zag fashion between circumferentially adjacent CAFs 2440. For example, first linking strut 2422a may be directly attached to a CAF 2440, for example at or near an axial midpoint thereof. First linking strut 2422a may connect to second linking strut 2422b, with the connection forming an apex pointing in the outflow direction. Second linking strut 2422b may connect to third linking strut 2422c, with the connection forming an apex pointing in the inflow direction. Third linking strut 2422c may connect to fourth linking strut 2422d, with the connection forming an apex pointing in the outflow direction. Finally, fourth linking strut 2422d may attach directly to one of the CAFs 2440, for example at or near an axial midpoint thereof. The connection between the second linking strut 2422b and the third linking strut 2422c may also serve as the connection to the outflow apex of an elongated cell 2417.

One of the benefits of the design of frame 2400 is that the frame 2400 is designed to have maximal space for coronary access. In particular, this may be achieved, at least in part, by the combination of the large, elongated cells 2417 on the outflow area of the frame in conjunction with an alternating smaller cell 2416 design structure.

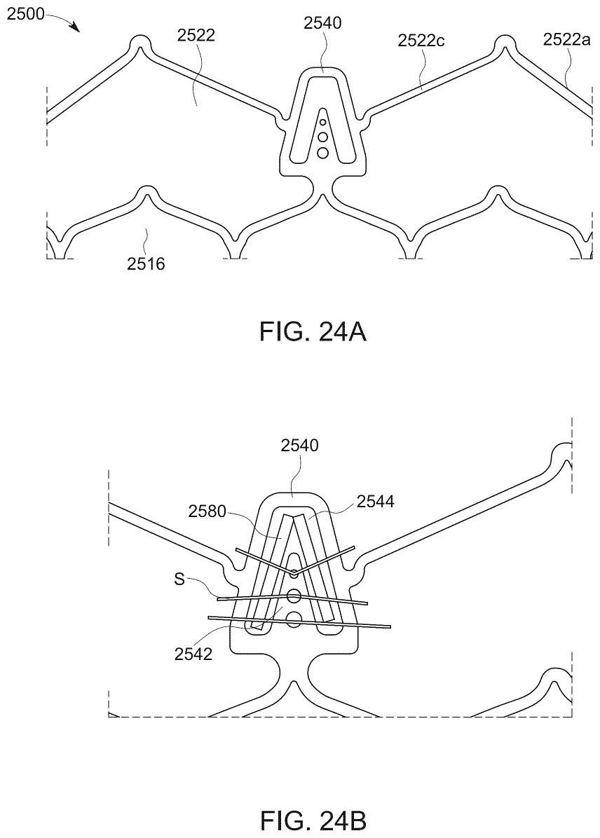

FIG. 24A illustrates another example of a portion of a frame 2500. Frame 2500 may be similar or identical to frames 1200, 1400, 1500, or 1600, with the exception of the CAF 2540. In the limited portion of the frame 2500 shown in FIG. 24A, outflow cells 2516 are shown, as well as linking struts 2522a, 2522c that at least partly form large cells 2522. Other than CAF 2540, which is described in greater detail below, the remainder of frame 2500 may have the features of any of frames 1200, 1400, 1500 or 1600 (or other frames described herein).

FIG. 24B shows an enlarged view of one of the CAF 2540, illustrating additional features regarding the connection of prosthetic leaflets to the CAF 2540. In the illustrated embodiment, the CAFs 2540 are integrally formed with the remainder of the frame, with the bottom (or inflow) ends of the CAFs 2540 attached to an outflow apex of an outflow cell 2516, and have side edges that are connected to linking struts 2522c at or near an axial midpoint of the CAF 2540. Each CAF 2540 may have an outer boundary that is generally triangular or trapezoidal. In other words, CAF 2540 may have a bottom or inflow edge or portion that may be referred to as a straight base with a relatively large length, and a top or outflow edge or portion that may be referred to as a straight top with a relatively small length. The base and top may be coupled by two angled side portions that extend at non-parallel angles to each other. In addition to this outer triangular or trapezoidal shape, a generally triangular projection 2542 may extend from the base toward the top of the CAF 2540, without actually contacting the top of the CAF 2540. One or more eyelets may be formed in the triangular projection 2542. In the illustrated embodiment, three eyelets are provided (in decreasing size from bottom to top) in a single column, but it should be understood that more or fewer eyelets (including no eyelets) may be provided, in the same or different arrangement, and with the same or different sizes as each other. With the configuration of CAF 2540 described above, a generally “A” or upside-down “V”-shape slot 2544 is formed within the CAF 2540.

Still referring to FIG. 24B, two leaflet tabs 2580 (e.g. the side edges or side extensions of two adjacent prosthetic leaflets that come together to form a prosthetic commissure) are positioned within and/or through the angled slot 2544 so that the leaflet tabs extend along angles that are non-parallel to the central longitudinal axis of the frame 2500. To help secure the leaflets tabs 2580 to the CAF 2540, one or more sutures S may be passed through the eyelets in the triangular extension 2542, through (and/or around) the leaflet tabs 2580, and wrap around the sides of the CAF 2540. One of the benefits of the design of CAF 2540 is that it is designed for angled leaflet attachment. When attaching prosthetic leaflets to the prosthetic heart valve (e.g. to the frame and/or fabric/tissue members on the frame), it is generally important to minimize prosthetic leaflet stress (and thus leaflet fatigue). The angled leaflet embodiment shown and described in connection with FIGS. 24A-B may allow for carefully tuning the angle of the leaflet attachment itself, and thus tuning the amount of stress/fatigue that the prosthetic leaflet may encounter during normal operation.

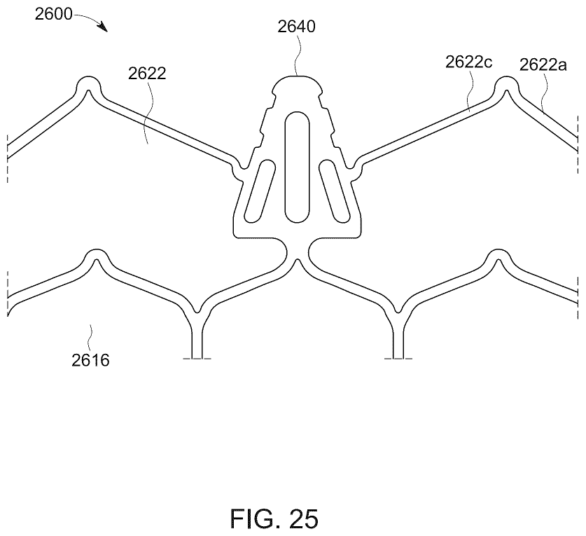

FIG. 25 illustrates another example of a portion of a frame 2600. Frame 2600 may be similar or identical to frames 1200, 1400, 1500, or 1600, with the exception of the CAF 2640. In the limited portion of the frame 2600 shown in FIG. 25, outflow cells 2616 are shown, as well as linking struts 2622a, 2622c that at least partly form large cells 2622. Other than CAF 2640, which is described in greater detail below, the remainder of frame 2600 may have the features of any of frames 1200, 1400, 1500 or 1600 (or other frames described herein).

In the illustrated embodiment, the CAFs 2640 are integrally formed with the remainder of the frame, with the bottom (or inflow) ends of the CAFs 2640 attached to an outflow apex of an outflow cell 2616, and have side edges that are connected to linking struts 2622c at or near an axial midpoint of the CAF 2640. Each CAF 2640 may have an outer boundary that is generally triangular or trapezoidal. In other words, CAF 2640 may have a bottom or inflow edge or portion that may be referred to as a straight base with a relatively large length, and a top or outflow edge or portion that may be referred to as a straight or curved top with a relatively small length. The base and top may be coupled by two angled side portions that extend at non-parallel angles to each other. One or more openings or eyelets may be formed in the CAF 2640. In the illustrated embodiment, an elongated central opening is provided that extends most of the axial height of the CAF 2640, and which may receive leaflet tabs therethrough. Two angled openings may be provided on either side of the elongated central opening, and the angled openings may be configured to receive sutures or leaflet tabs therethrough to assist with leaflet and/or cuff attachment to the frame 2600. In this embodiment, the tapered design of the CAF 2640 may help to enable a smaller collapsed profile of the frame 2600.

The frames shown in FIGS. 4A and 6-25 include various combinations of components and features shown in specific examples. However, it should be understood that features of these frames may be mixed and matched where appropriate. For example, the inflow sections of any of these frames may generally be interchangeable to arrive at specific combinations not actually shown. Similarly, the outflow sections of any of these frames may generally be interchangeable to arrive at specific combinations not actually shown. Further, the specific CAFs shown may be interchangeable with other CAFs shown herein. Other specific features of the frames may be modified as appropriate, even though not specifically shown. For example, the number and configuration of the linking struts in the outflow section of any of these frames may be replaced with that of other frames shown herein. The position of the CAF to which the linking strut(s) attach (e.g. top, middle, or bottom) may be changed as desired in any of these frames. The linking struts may all have the same width as each other, or may be provided with different widths, including decreasing width in a direction toward the CAF. Although adjacent linking struts are shown in embodiments with certain angles being formed between them when in the expanded condition, it should be understood that other angles may instead be provided, including those seen on other embodiments of frames herein. The number of cells per row in the inflow sections, the number of rows within the inflow section, and the specific shape of the cells in the inflow section, may be adjusted. For example, the inflow section of any of the frames disclosed herein may be swapped out for the inflow section of any of the other frame disclosed herein. As noted in connection with FIG. 5, any of the frames disclosed herein may include strut widths that are generally the same, or that have a gradient, including for example relatively large strut widths nearer the inflow end of the frame and generally narrower struts in the direction toward the outflow end of the frame. Also, as explained in connection with FIG. 5, any of the frames disclosed herein may include opening angles that are the same, or that are different, as desired. Further, any of the frames may be provided with eyelets at the inflow apices of the inflow cells, or instead of eyelets notches, or neither eyelets nor notches, or other features that serve a similar purpose as the eyelets or notches. Put simply, the various frame embodiments described herein are provided with specific embodiments that have individual features that may provide a particular benefit, and a person skilled in the art should understand that different features of different embodiments may be combined (including in manners not explicitly shown herein) to achieve a frame that includes the benefits of each individual feature incorporated into the frame.

Further, in all of the embodiments disclosed herein, when the prosthetic heart valve that incorporates the particular frame described herein is in the expanded condition, the frame may be generally cylindrical, or otherwise have a substantially constant outside diameter, without significant flaring of the type that may be found in self-expanding valves. Further, all of the large outflow cells (e.g. 522, 722, 822 etc.) are shown as lacking any axial strut, with the exception of frame 1700 and frame 2200. It should be understood that, in this context, a CAF that forms part of the large outflow cell is not an axial strut. With all of the large outflow cells (e.g. 522, 722, 822 etc.) described herein, the outflow cells (and/or the open space defined by the outflow cells) lack symmetry (both unilateral and bilateral symmetry). Particularly in the embodiments that lack axial struts that form the large outflow cells (e.g. 522, 722, 822 etc.), the outflow edge of the frame may include relatively deep valleys, particularly in areas between adjacent CAFs. These deep valleys may be intentionally provided and may be generally configured to be positioned below (e.g. upstream) of the coronary arteries upon implantation, allowing for a high level of post-procedural coronary access.

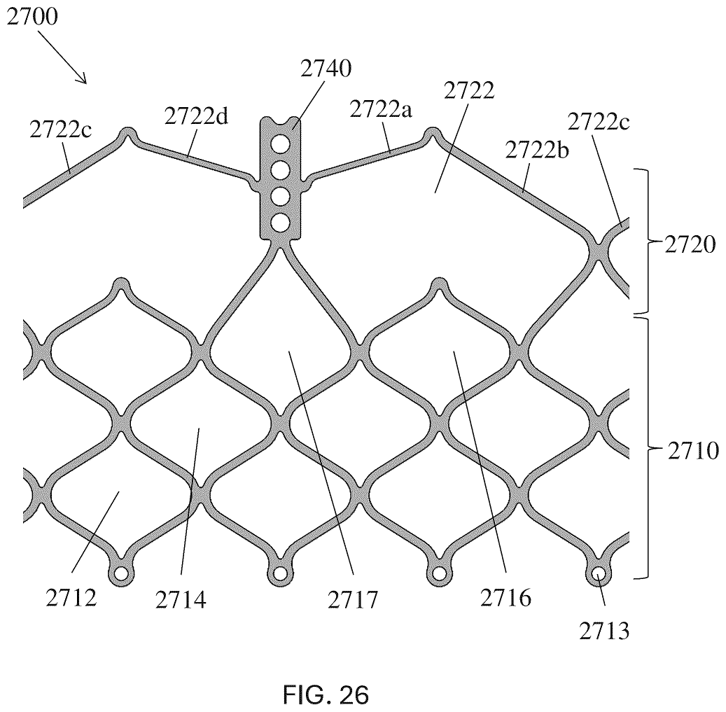

As noted above, various features of the different frame examples shown and described herein may be combined to achieve a desired result. One particular example is shown in FIG. 26, in which a portion of a frame 2700 is shown which combines features similar to those of frame 1400 of FIG. 13 with features similar to those of frame 2400 of FIG. 23. In this example, FIG. 26 shows about one-third of the frame 2700 (assuming the frame is being used with three prosthetic leaflets). As with other examples described herein, if frame 2700 is being used for a prosthetic heart valve with three prosthetic leaflets, the portion of frame 2700 shown in FIG. 26 would repeat about three times in a generally circular or annular shape. Frame 2700 may include an inflow section 2710 and an outflow section 2720. The inflow section 2710 may have an inflow-most row of cells 2712, an adjacent intermediate row of cells 2714, and an outflow row of cells 2716. In the illustrated embodiment, cells 2712, 2714, and 2716 are all substantially diamond-shaped. Rows 2712 and 2714 may be provided in complete rows of identical cells, whereas row 2716 may include elongated cells 2717 (which may have a diamond-shape or kite-shape) that alternate with diamond-shaped cells 2716. Elongated outflow cells 2717 are provided, in the illustrated embodiment, directly below each CAF 2740 (which may be similar or identical to CAFs 1440), as well as directly below the connection of linking struts 2722b, 2722c (described in greater detail below), for a total of six diamond-shaped cells 2716 and six elongated outflow cells 2717 when frame 2700 is used as part of a three-leaflet valve. Each elongated cell 2717 may extend an axial distance toward the outflow end that is greater than the axial extent of the diamond-shaped cells 2716. In the illustrated embodiment, the inflow apices of the inflow cells 2712 may include eyelets 2713, which may be similar or identical to eyelets 513.

In the illustrated example, four linking struts 2722a-d are provided in a zig-zag fashion between circumferentially adjacent CAFs 2740. For example, first linking strut 2722a may be directly attached to a CAF 2740, for example at or near an axial midpoint thereof. First linking strut 2722a may connect to second linking strut 2722b, with the connection forming an apex pointing in the outflow direction. Second linking strut 2722b may connect to third linking strut 2722c, with the connection forming an apex pointing in the inflow direction. Third linking strut 2722c may connect to fourth linking strut 2722d, with the connection forming an apex pointing in the outflow direction. Finally, fourth linking strut 2722d may attach directly to one of the CAFs 2740, for example at or near an axial midpoint thereof. The connection between the second linking strut 2722b and the third linking strut 2722c may also serve as the connection to the outflow apex of an elongated cell 2717.

One of the benefits of the design of frame 2700, similar to frame 2400, is that the frame 2700 may be designed to have maximal space for coronary access. In particular, this may be achieved, at least in part, by the combination of the large, elongated cells 2717 on the outflow area of the frame in conjunction with an alternating smaller cell 2716 design structure. Another benefit of the design of frame 2700 may be that the relatively narrow angle formed by the two outflow struts in elongated cells 2717 (compared to the relatively wide angle formed by the two outflow struts in adjacent cells 2716) may help to allow for the CAF 2740 to deflect during use of the prosthetic heart valve. In other words, as the prosthetic leaflets (which are attached to the CAFs 2740) close to prevent backflow through the prosthetic heart valve incorporating frame 2700, the force of the prosthetic leaflets resisting backflow may cause the CAFs 2740 to slightly deflect inwardly. This deflection may be desirable to help reduce stress on the prosthetic leaflets, which may be particularly important in balloon-expandable prosthetic heart valves, for example if frame 2700 is formed of stainless steel or a cobalt chrome material, as those materials are typically quite rigid (and rigid materials may not easily dampen forces and/or stress/strain being placed on the prosthetic leaflets). Further, although frame 2700 is shown as having an inflow section 2710 with three rows of cells (2712, 2714 and the row of alternating cells 2716/2717), in some examples the inflow section 2710 may be provided with only two rows of cells. For example, if a prosthetic heart valve incorporating frame 2700 is provided in different sizes (e.g. to account for patients having differently sized annuluses), the larger sizes may be provided with three rows of cells in the inflow section 2710 as shown in FIG. 26, but the smaller sizes may omit one of the inflow rows of cells, such as row of cells 2714, to accommodate a smaller native anatomy. As with other embodiments described herein, it should be understood that a prosthetic heart valve that incorporates frame 2700 may include an inner skirt and/or an outer skirt, which may be similar to or different than inner skirt/cuff 260 and outer skirt/cuff 270.

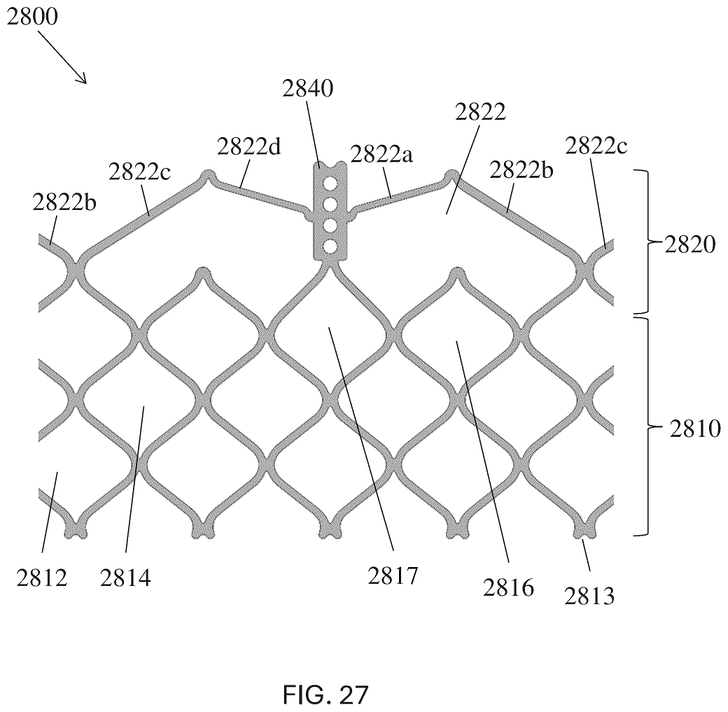

FIG. 27 shows slightly more than one-third of another frame 2800 (assuming the frame is being used with three prosthetic leaflets). Frame 2800 may be identical to frame 2700 with certain exceptions, and thus only the exceptions are described here, and it should be understood that other features not described may be identical to those described in connection with frame 2700 of FIG. 26. For example, similar to frame 2700, frame 2800 may include an inflow section 2810 and an outflow section 2820, the inflow section 2810 having three rows of cells 2812, 2814, and 2816 (with cells 2817 alternating with cells 2816). Cells 2812 and 2814 may be substantially identical to cells 2712 and 2714, although cells 2812 are shown with notches 2813 instead of eyelets 2713. The other main difference is that cells 2816 and 2817 may be substantially identical to each other, although cells 2817 may be very slightly elongated in the outflow direction compared to cells 2816, but if there is any elongation, it is minimal. Otherwise, the outflow section 2820, including cells 2822 and four linking struts 2822a-d connecting each circumferentially adjacent pair of CAFs 2840, is generally the same as in frame 2700. However, because cells 2816 and 2817 are identical (or nearly identical) in shape, the smaller elongation of cells 2817 compared to cells 2717 results in smaller open space of the hybrid cells 2822, which may lead to smaller amounts of coronary access.

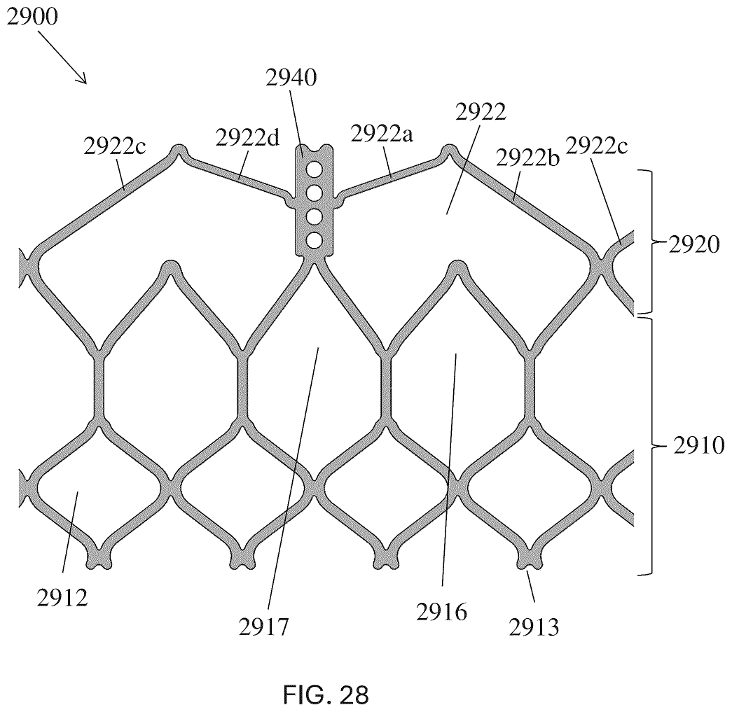

FIG. 28 shows slightly more than one-third of another frame 2900 (assuming the frame is being used with three prosthetic leaflets). Frame 2900 may be identical to frame 2700 with certain exceptions, and thus only the exceptions are described here, and it should be understood that other features not described may be identical to those described in connection with frame 2700 of FIG. 26. For example, similar to frame 2700, frame 2900 may include an inflow section 2910 and an outflow section 2920. However, the inflow section 2910 is shown with two rows of cells, including an inflow row 2912 (which includes notches 2913 at the inflow apex instead of eyelets) of diamond-shaped cells, and a row of enlarged outflow cells 2916, 2917. Each outflow cell 2916, 2917 may be longer axially than all of the inflow cells 2912, and for example include two inflow struts forming an apex pointing toward the inflow end, two outflow struts forming an apex pointing toward the outflow end, and two axial struts connecting the pairs of inflow and outflow struts. With this configuration, the axial extent of the inflow section 2910 may be generally similar to the axial extent of the inflow section 2710 of frame 2700, although inflow section 2910 is shown with two rows of cells while inflow section 2710 is shown with three rows of cells. Outflow cells 2916 and 2917 may be substantially identical in shape to each other, although in some examples the outflow cells 2917 may be slightly elongated axially in the outflow direction (although they need not be) compared to outflow cells 2916. Otherwise, the outflow section 2920, including cells 2922 and four linking struts 2922a-d connecting each circumferentially adjacent pair of CAFs 2940, is generally the same as in frame 2700. However, because cells 2916 and 2917 are identical (or nearly identical) in shape, the configuration may result in smaller open space of the hybrid cells 2922, which may lead to smaller amounts of coronary access.

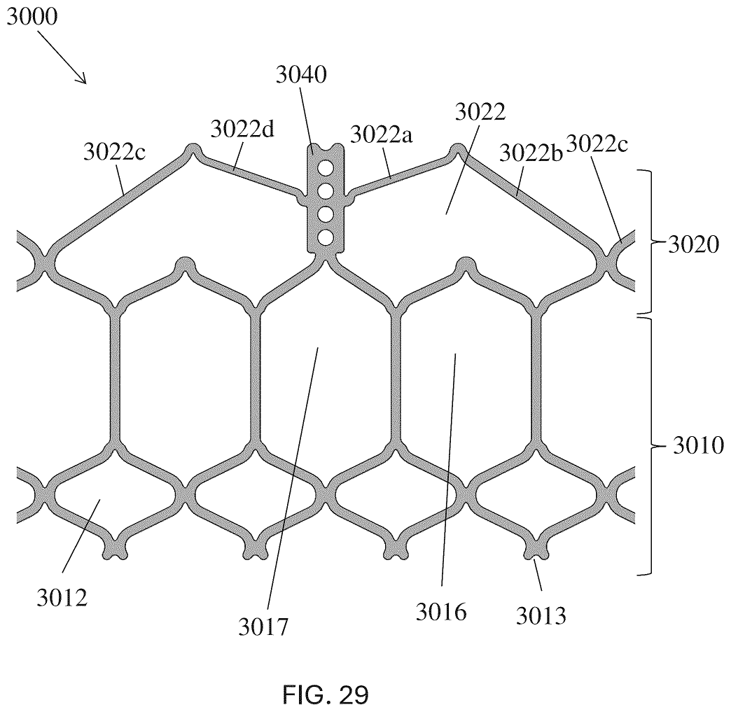

FIG. 29 shows slightly more than one-third of another frame 3000 (assuming the frame is being used with three prosthetic leaflets). Frame 3000 may be identical to frame 2900 with certain exceptions, and thus only the exceptions are described here, and it should be understood that other features not described may be identical to those described in connection with frame 2900 of FIG. 28. For example, similar to frame 2900, frame 3000 may include an inflow section 3010 having two rows of cells and an outflow section 3020 having one row of cells. For example, the inflow section 3010 may include an inflow row 3012 (which includes notches 3013 at the inflow apex) of diamond-shaped cells, and a row of enlarged outflow cells 3016, 3017. Each outflow cell 3016, 3017 may be longer axially than all of the inflow cells 3012, and for example include two inflow struts forming an apex pointing toward the inflow end, two outflow struts forming an apex pointing toward the outflow end, and two axial struts connecting the pairs of inflow and outflow struts. With this configuration, the axial extent of the inflow section 3010 may be generally similar to the axial extent of the inflow section 2710 of frame 2700, although inflow section 3010 is shown with two rows of cells while inflow section 2710 is shown with three rows of cells. Outflow cells 3016 and 3017 may be substantially identical in shape to each other, although in some examples the outflow cells 3017 may be slightly elongated axially in the outflow direction (although they need not be) compared to outflow cells 3016. The main difference between frame 2900 and frame 3000 is that diamond-shaped inflow cells 3012 may be slightly compressed in the axial direction compared to inflow cells 2912, and the axial struts of outflow cells 3016, 3017 may be longer than the axial struts of outflow cells 2916, 2917. With this configuration, the inflow and outflow pairs of struts of outflow cells 3016, 3017 may form a larger opening angle compared to the corresponding struts of cells 2916, 2917. Otherwise, the outflow section 3020, including cells 3022 and four linking struts 3022a-d connecting each circumferentially adjacent pair of CAFs 3040, is generally the same as in frame 2900, although the hybrid cells 3022 may have even less open area compared to hybrid cells 2922.

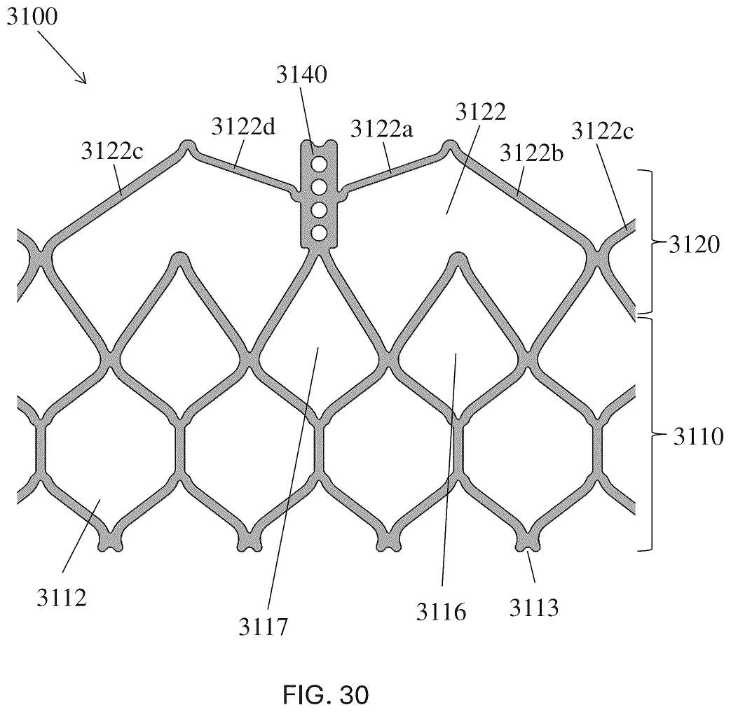

FIG. 30 shows slightly more than one-third of another frame 3100 (assuming the frame is being used with three prosthetic leaflets). Frame 3100 may be generally similar to frames 2900 and 3000 with certain exceptions, and thus only the exceptions are described here, and it should be understood that other features not described may be identical to those described in connection with frames 2900 or 3000 of FIG. 28 or 29. For example, similar to frame 3000, frame 3100 may include an inflow section 3110 having two rows of cells and an outflow section 3120 having one row of cells. For example, the inflow section 3110 may include an inflow row of cells 3112 (which include notches 3113 at the inflow apex) of cells. Inflow cells 3112 may include a pair of struts forming an apex pointing in the inflow direction, a pair of struts forming an apex pointing in the outflow direction, and a pair of axial struts connecting the two pairs of struts. With this configuration, cells 3112 are axially longer compared to cells 3012, and form more of a hexagon shape than a diamond shape. The outflow row of cells 3116, 3117 may be substantially diamond-shaped of kite-shaped. With this configuration, the axial extent of the inflow section 3110 may be generally similar to the axial extent of the inflow section 2710 of frame 2700, although inflow section 3110 is shown with two rows of cells while inflow section 2710 is shown with three rows of cells. Outflow cells 3116 and 3117 may be substantially identical in shape to each other, although in some examples the outflow cells 3117 may be slightly elongated axially in the outflow direction (although they need not be) compared to outflow cells 3116. The outflow section 3120, including cells 3122 and four linking struts 3122a-d connecting each circumferentially adjacent pair of CAFs 3140, is generally the same as in frame 3000, although the hybrid cells 3122 may have slightly more open area compared to hybrid cells 3022 (e.g., similar to the amount of open space in hybrid cells 2922).

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve, comprising:
a balloon-expandable frame extending between an inflow end and an outflow end;
a plurality of prosthetic leaflets mounted within the frame; and
an inner skirt positioned between the plurality of prosthetic leaflets and the frame;
wherein the frame includes a first row of diamond-shaped cells at the inflow end of the frame, a second row of diamond-shaped cells, and a row of outflow cells positioned at the outflow end of the frame,
wherein each cell in the row of outflow cells is not diamond-shaped, and, in an expanded condition of the frame, each cell in the row of outflow cells defines an interior area that is larger than an interior area defined by each cell in the first and second rows of diamond-shaped cells,
wherein each cell in the row of outflow cells is at least partially defined by a commissure attachment feature, and each cell in the row of outflow cells lacks symmetry.

2. The prosthetic heart valve of claim 1, wherein the frame includes a third row of diamond-shaped cells positioned between the first and second rows of diamond-shaped cells.

3. The prosthetic heart valve of claim 1, wherein the first and second rows of diamond-shaped cells include twelve cells each, and the row of outflow cells includes six cells.

4. The prosthetic heart valve of claim 1, wherein the frame includes an inflow section that includes the first and second rows of diamond-shaped cells, and an outflow section that includes the row of outflow cells, such that more force is required to expand the inflow section than the outflow section.

5. The prosthetic heart valve of claim 1, wherein the commissure attachment feature includes a plurality of commissure attachment features that each have a rectangular or triangular shape, each of the commissure attachment features being attached to one cell in the second row of diamond-shaped cells, and defining portions of two cells in the outflow row of cells.

6. The prosthetic heart valve of claim 5, wherein the one cell in the second row of diamond-shaped cells includes two struts forming an outflow apex that is coupled to an inflow end of the corresponding commissure attachment feature.

7. The prosthetic heart valve of claim 5, wherein each cell in the row of outflow cells is defined by (i) a portion of one of the plurality of commissure attachment features, (ii) struts at an outflow end of a multiplicity of cells in the second row of diamond-shaped cells, and (iii) a plurality of linking struts extending between the outflow end of one of the multiplicity of cells in the second row of diamond-shaped cells and the one of the plurality of commissure attachment features.

8. The prosthetic heart valve of claim 7, wherein the plurality of linking struts including a first linking strut extending in an outflow direction away from the one of the multiplicity of cells in the second row of diamond-shaped cells, a second linking strut extending from the first linking strut in a direction back toward the second row of diamond-shaped cells, and a third linking strut extending from the second linking strut towards, and connecting to, the one of the plurality of commissure attachment features.

9. The prosthetic heart valve of claim 8, wherein the third linking strut is coupled to an outflow end of the one of the plurality of commissure attachment features.

10. The prosthetic heart valve of claim 8, wherein the first linking strut has a width that is greater than a width of the second linking strut.

11. A prosthetic heart valve, comprising:
a balloon-expandable frame extending between an inflow end and an outflow end; and
a plurality of prosthetic leaflets mounted within the frame;
wherein the frame includes a first row of kite-shaped cells, and a second row of kite-shaped cells positioned in an outflow direction relative to the first row of kite-shaped cells,
wherein each cell in the first row is defined by two inflow struts and two outflow struts, each cell in the second row of kite-shaped cells is defined by two inflow struts and two outflow struts, the two outflow struts in the first row being the same as the two inflow struts in the second row.

12. The prosthetic heart valve of claim 11, wherein the two inflow struts of each cell in the first row are wider than the two outflow struts of each cell in the first row or the two outflow struts of each cell in the second row.

13. The prosthetic heart valve of claim 12, wherein the two outflow struts of each cell in the first row are wider than the two outflow struts of each cell in the second row.

14. The prosthetic heart valve of claim 11, wherein the two inflow struts of each cell in the first row are thicker than the two outflow struts of each cell in the first row or the two outflow struts of each cell in the second row.

15. The prosthetic heart valve of claim 14, wherein the two outflow struts of each cell in the first row are thicker than the two outflow struts of each cell in the second row.

16. The prosthetic heart valve of claim 11, wherein the two inflow struts in each cell in the first row form a first angle relative to each other, and the two outflow struts in each cell in the first row form a second angle relative to each other, the first angle being larger than the second angle.

17. The prosthetic heart valve of claim 16, wherein the two inflow struts in each cell in the second row form a third angle relative to each other, and the two outflow struts in each cell in the second row form a fourth angle relative to each other, the third angle being larger than the fourth angle, and the first angle being larger than the third angle.

18. A method of implanting a prosthetic heart valve, the method comprising:
advancing a distal end of a delivery device to a native aortic valve while the prosthetic heart valve is crimped over a balloon on the distal end of the delivery device while the balloon is in an uninflated condition;
confirming, under fluoroscopy, that an inflow end of a frame of the prosthetic heart valve is aligned with a desired target site while the balloon is in the uninflated condition; and
after confirming, passing inflation media into the balloon to inflate the balloon to expand the prosthetic heart valve into the native aortic valve;
wherein after expanding the prosthetic heart valve, the inflow end of the frame is positioned at the desired target site and wherein, between confirming and expanding the prosthetic heart valve, the delivery device is not translated relative to the native aortic valve.

19. The method of claim 18, wherein, during expanding the prosthetic heart valve, the frame of the prosthetic heart valve axially foreshortens.

20. The method of claim 19, wherein, during expanding the prosthetic heart valve, an outflow end of the frame translates toward the target site while the inflow end of the frame does not translate relative to the target site.

21. The method of claim 18, wherein as the balloon inflates to expand the prosthetic heart valve, there is greater friction between the balloon and an inflow end of the prosthetic heart valve than there is between the balloon and an outflow end of the prosthetic heart valve.
